(19) United States
(12) Patent Application Publication
(10) Pub. No.: US 2016/0345954 A1
(43) Pub. Date: Dec. 1, 2016

(54) Anchor Devices and Methods of Use

(71) Applicants: James F. Marino, La Jolla, CA (US); Jamil Elbanna, San Diego, CA (US)

(72) Inventors: James F. Marino, La Jolla, CA (US); Jamil Elbanna, San Diego, CA (US)

(21) Appl. No.: 15/163,151

(22) Filed: May 24, 2016

Related U.S. Application Data
(60) Provisional application No. 62/167,191, filed on May 27, 2015.
(60) Provisional application No. 62/232,295, filed on Sep. 24, 2015.

(51) Int. Cl. A61B 17/04

(52) U.S. Cl. CPC A61B 17/0401; A61B 2017/0412; A61B 2017/042; A61B 2017/0438; A61B 2017/0496

(57) ABSTRACT

The method of fixing soft tissue to bone includes placing an anchor device superficial to a segment of soft tissue superficially juxtaposed to a unicortical blind hole in a bone, the anchor device having a relaxed state characterized by a first outer dimension. The method includes driving the soft tissue segment into the blind hole using the anchor device. The anchor device transiently constricts to a constricted state during insertion through the blind hole, the constricted state characterized by a second outer dimension that is smaller than the first outer dimension. The method includes trapping a portion of the soft tissue segment between a portion of the anchor device and a portion of the bone when at least a portion of the anchor device is disposed sub-cortically and relaxes towards the relaxed state. Related devices, systems and methods are also described.

### CROSS-REFERENCE TO PRIORITY DOCUMENTS

This application claims priority under 35 U.S.C. §119(e) U.S. Provisional Patent Application Ser. No. 62/167,191; filed May 27, 2015, entitled “Anchor Devices and Methods of Use,” and U.S. Provisional Patent Application Ser. No. 62/232,295; filed Sep. 24, 2015, entitled “Anchor Devices and Methods of Use,” which are incorporated by reference herein in their entireties.

### BACKGROUND

In orthopedic surgical procedures, surgeons attach or reattach soft tissue structures to bone using anchor devices. Difficulties can arise from overly complicated anchor devices and time consuming procedures to implant that can still ultimately result in failure of the attachment.

### SUMMARY

In one aspect, disclosed is a method of fixing soft tissue to bone. The method includes placing an anchor device superficial to a segment of soft tissue superficially juxtaposed to a unicortical blind hole in a bone, the anchor device having a relaxed state characterized by a first outer dimension. The method includes driving the soft tissue segment into the blind hole using the anchor device. The anchor device transiently constricts to a constricted state during insertion through the blind hole, the constricted state characterized by a second outer dimension that is smaller than the first outer dimension. The method includes trapping a portion of the soft tissue segment between a portion of the anchor device and a portion of the bone when at least a portion of the anchor device is disposed sub-cortically and relaxes towards the relaxed state.

The anchor device can be fabricated from one or more biocompatible polymers. The biocompatible polymers can include Polyetheretherketone (PEEK) and ultrahigh molecular weight polypropylene (UHMWPE). The anchor device can be fabricated from one or more absorbable biocompatible polymers. The absorbable biocompatible polymers can include polyglycolic acid (PGA), polylactic acid (PLA), polydioxanone, caprolactone, or a combination thereof. The soft tissue can be a tendon or a ligament.

In an interrelated aspect, disclosed is an anchor device for attaching a soft tissue segment within bone having a body having a distal end region and a proximal end region each at least partially surrounding an interior volume forming a slot extending generally along a longitudinal axis of the body. The anchor device has a concave saddle surface at the distal end region of the body configured to receive and redirect a soft tissue segment extending through the slot in a first direction from the proximal end region of the body to a second direction that is at an angle to the first direction.

The distal end region of the body can be configured to be positioned sub-cortically to a bone defect and at least a portion of the proximal end region of the body can be configured to be positioned intra-cortically within the bone defect. During insertion of the anchor device into the bone defect the body can be configured to transiently constrict from a relaxed state having a first outer dimension to a constricted state having a second outer dimension that is smaller than the first outer dimension. The soft tissue segment can be a tendon or a ligament. At least a portion of the soft tissue segment can be trapped between a portion of the anchor device and a portion of the bone defect when at least a portion of the anchor device can be disposed sub-cortically and relaxes towards the relaxed state. The anchor device can be fabricated from one or more biocompatible polymers, including Polyetheretherketone (PEEK) and ultrahigh molecular weight polypropylene (UHMWPE). The anchor device can be fabricated from one or more absorbable biocompatible polymers, including polyglycolic acid (PGA), polylactic acid (PLA), polydioxanone, caprolactone, or a combination thereof.

In an interrelated aspect, disclosed is an anchor device for attaching a soft tissue segment within bone having a cortex and a sub-cortical region having a body having a distal end region and a proximal end region each at least partially surrounding an interior volume forming a slot extending generally along a longitudinal axis of the body. The body is configured to be transiently constricted by a cortical aperture within the bone from a relaxed state having a first outer dimension to a constricted state having a second outer dimension that is smaller than the first outer dimension such that the soft tissue segment is trapped between at least a portion of the body and at least a portion of the bone cortex upon subcortical relaxation of the body towards the relaxed state.

The anchor device can further comprise a concave saddle surface at the distal end region of the body configured to receive and redirect the soft tissue segment extending through the slot in a first direction from the proximal end region of the body to a second direction that is at an angle to the first direction. A pair of opposed prongs can extend from the distal end region of the body on either side of the midline axis and defining the concave saddle surface. The slot can provide a generally c-shaped cross-section to at least a portion of the body. The anchor device can include a proximally-extending cortical feature near the proximal end region of the body, the cortical feature having an arcuate length and cross-sectional size that is smaller than an arcuate length and cross-sectional size of the proximal end region of the body forming a cortical rim retention shelf near the proximal end region of the body. The body can have a tapered sidewall between the cortical rim retention shelf to the distal end region of the body. The cortical rim retention shelf can contract radially around the longitudinal axis when in the constricted state. At least a portion of the anchor device can flex in an arcuate manner when in the constricted state. The cortical rim retention shelf can further include a proximal aspect forming an edge having a plurality of spikes, serrations, or projecting elements. The proximal aspect can capture soft tissue between the proximal end region of the device and at least a portion of bone through which the device is implanted. The body can have monolithic and have a substantially conical or frusto-conical shape. The body can be generally pliable and elastic and have a transverse cross-sectional geometry that is generally arcuate-shaped forming a concave surface and a convex surface to the body. An arcuate length of the transverse cross-section near the distal end region of the body can be shorter than an arcuate length of the transverse cross-section near the proximal end region of the body. When in use within a bored defect within the bone, the distal end region of the body can be positioned within a depth of the medullary cavity of the bone and the proximal end region of the body can be positioned within the bone near a cortical surface of the bored defect. The soft tissue segment can be a tendon or a ligament having an attached end and a detached end. The soft tissue segment can be positioned along the longitudinal axis of the body coursing along both the concave and the convex surfaces of the body such that soft tissue segment is sharply redirected by wrapping around the distal end region of the body and the detached end is compressed between the convex surface of the body near the proximal end region and the cortical surface of the bored defect. The arcuate length of the transverse cross-section near the proximal end region of the body can be greater than a diameter of the bored defect.

In an interrelated aspect, disclosed is an anchor device for attaching materials within bone having a body having a distal end region, a proximal end region, and a plurality of struts extending between the distal end region to the proximal end region and at least partially surrounding an interior volume of the body. The anchor device has an attachment feature positioned within the interior volume of the body and coupled near the distal end region, the attachment feature configured to secure material to the body. Upon removal of a constraint and after delivery of the anchor device into bone, the body passively transitions from a constrained, delivery configuration that is radially contracted and axially elongated to a relaxed, deployment configuration that is radially expanded and axially shortened.

The material secured by the attachment feature to the anchor device can be suture or cable material. The material can be further affixed to a soft tissue structure such as a tendon, ligament, and joint capsule. The attachment feature can be an element such as a suture anchor element, a cleat element, a post, a saddle-shaped element, a pulley, and a crimping element. The attachment feature can include a post extending transverse to the longitudinal axis of the body. The attachment feature can include a saddle shaped element to which the material is secured. The attachment feature can include a cleat element to secure the material. The cleat element can employ a cam action or ratcheting reel assembly to progressively tension the material and approximate the material to the proximal end region of the body. The cleat element can include at least one suture anchor element, two apertures and an intervening central post. A first portion of the material can overlap a second portion of the material resulting in a unidirectional tensioning mechanism of the material with the cleat element. The cleat element can have at least two suture anchor elements, each having an aperture configured to allow the material to extend through. Applying tension to the material can force the at least two suture anchor elements to form a splayed configuration. The tension applied to the material can be maintained by the at least two suture anchor elements. At least a part of the material passed through the apertures of the at least two suture anchor elements and wrapped around the commonly formed post can result in a portion of the material overlapping another portion of the material. The attachment feature can include a crimping element to secure the material. The material can be attached to a soft tissue structure. The material can be secured with an interference pin delivered through an opening in the proximal end region of the body. A proximal aspect of the crimping element can include a cable or suture transecting feature. Any of the anchor devices described herein can include a penetrating tip coupled to the distal end region of the body. The penetrating tip can have a trephine, fluted or conically-tapered outer geometry to facilitate penetration of bone. The material can include a tensioning element configured to approximate the distal end region and the proximal end region upon application of tension on the material causing the plurality of struts to radially expand. The penetrating tip and the tensioning element can be integrated with the attachment feature forming an inner body extending within the internal volume and surrounded at least in part by the body.

The proximal end region can include a discontinuous outer wall defining a proximal opening to the interior volume of the body within which the material is disposed such that soft tissue affixed to the material is in direct contact with the bone. The plurality of struts can expand near the proximal end region to a greater degree than the plurality of struts expands near the distal end region. The body of the anchor device can be fabricated from a superelastic metal. The constraint can include a generally rigid tubular element. The generally rigid tubular element can include a slot to accommodate the material. The constraint can include a circumferential ring element. The circumferential ring element can be positioned between the material and the bone thereby preventing abrasion of the material, the material being suture, cable or soft tissue. The constraint can include a primary constraint and a secondary constraint. The secondary constraint can be positioned over at least a region of the primary constraint during storage of the anchor device.

In an interrelated aspect, disclosed is a suture or soft tissue anchor device having a self-expanding device configured to be constrained prior to delivery having a relatively reduced diameter with a relatively extended length, and subsequently expanded to a relatively larger diameter with a relatively shortened length after deployment and delivery within bone. The self-expanding device can include a generally cylindrical or truncated cylindrical shaped body having two or more slots configured to be constrained prior to delivery.

In an interrelated aspect, disclosed is a suture or soft tissue anchor device configured to employ a self-expanding device that is constrained prior to delivery, in a relatively reduced diameter and relatively extended length, with subsequent deployment and delivery within bone, where it is configured to expand to a relatively larger diameter with a relatively shortened length; in which suture material or cable attached to the soft tissue being approximated or secured to the bone anchor is affixed or otherwise associated with the distal terminus or an element associated with the distal terminus of the anchor.

In an interrelated aspect, disclosed is a suture or soft tissue anchor device configured to employ a self-expanding device that is constrained prior to delivery, in a relatively reduced diameter and relatively extended length, with subsequent deployment and delivery within bone, where it expands to a relatively larger diameter with a relatively shortened length; in which suture material or cable attached to the soft tissue being approximated or secured to the bone anchor is affixed or otherwise associated with the distal terminus or an element associated with the distal terminus of the anchor, such that tension applied on the suture or cable results in a force that foreshortens the length and expands the diameter of the anchor.

The anchor device can be fabricated from a superelastic metal, such as nitinol. A suture can be affixed to a soft tissue structure and secured to the distal end of the self-expanding implant. The soft tissue structure can be a tendon, ligament, or joint capsule. The self-expanding device can be further expanded by means of a tensioning element that approximates the distal and proximal ends of the device. The anchor device can further include a distal tip that is conically tapered to facilitate penetration of bone. The distal tip can be configured with trephine or fluted geometry to facilitate penetration of bone. A suture affixed to a soft tissue structure can be passed through an aperture located distally within the device and delivered through the proximal aperture of the device for subsequent tensioning. A cam action cleat mechanism can be used to progressively tension the sutures and approximate the attached soft tissue element to the proximal aspect of the self-expanding device. A ratcheting reel assembly can be used to tension the sutures and approximate the attached soft tissue structure to the proximal aspect of the self-expanding device. The sutures coursing within the device and attached to a soft tissue structure can be secured to the device with a crimping element. The proximal aspect of the crimping mechanism can include a cable or suture transecting feature. The sutures coursing within the device and attached to a soft tissue structure can be secured with an interference pin, delivered within the proximal aperture. The interference pin can have a tapered distal tip. The cable or sutures restrained by the interference pin can be transected immediately proximal or adjacent to the proximal aspect of the interference pin with a cable or suture cutter having a rotary actuation mechanism.

A generally tubular configured confinement element can be used to maintain the self-expanding device in its confined geometry prior to distal delivery out of the confinement tube and into the bone. The primary confinement tube can have a slot to accommodate introduction of suture material attached to a soft tissue structure. A secondary confinement tubing or ring element can be positioned over the distal segment of the primary confinement tube during storage and can be removed after chilling the self-expanding device immediately or shortly prior to deployment within the bone. A secondary confinement tubing can be positioned over the distal segment of the primary confinement tube during storage and can be removed after chilling the self-expanding device immediately or shortly prior to deployment within the bone.

The device can include at least one suture anchor element having two apertures and an intervening central post, to allow suture to extend through the apertures and around the central post such that a portion of the suture overlaps another portion of the suture, resulting in a unidirectional tensioning mechanism of the suture or cleat mechanism. The device can employ a cam action cleat mechanism to progressively tension the sutures and approximate the attached soft tissue element to the proximal aspect of the self-expanding device. The unidirectional tensioning mechanism can include at least two suture anchor elements, each having an aperture and a commonly formed post comprising at least one post element from each of the at least two suture anchor elements. The suture can be passed through the apertures of the at least two suture anchor elements and wrapped around the common post. The device can include at least two suture anchor elements, each having an aperture configured to allow suture to extend through. Applying tension to the suture can force the at least two suture anchor elements to form a splayed configuration. The tension applied to the suture can be maintained by the at least two suture anchor elements. At least a part of the suture passed through the apertures of the at least two suture anchor elements and wrapped around the commonly formed post, results in a portion of the suture overlapping another portion of the same suture.

In an interrelated aspect, disclosed is a self-expanding suture anchor device having a pre-deployment confined configuration which is at least in part maintained by a circumferential ring element. In the deployed state of the suture anchor device, the device serves to provide a suture abrasion protective function resulting from its surface features and deployment position, located between the suture material and the bone.

In an interrelated aspect, disclosed is an anchor device for attaching tissue within bone having a body having a distal end region, a proximal end region, and a plurality of struts extending between the distal end region to the proximal end region and at least partially surround an interior volume of the body. The body passively transitions from a constrained, delivery configuration that is radially contracted and axially elongated to a relaxed, deployment configuration that is radially expanded and axially shortened upon removal of a constraint on the plurality of struts after delivery into bone. The device includes an attachment feature positioned within the interior volume of the body near the distal end region. The attachment feature is configured to secure the tissue to the anchor device. The device includes a distal penetrating tip. The proximal end region includes a discontinuous outer wall defining a proximal opening to the interior volume of the body within which the secured tissue is disposed so as to be in direct contact with the bone.

In an interrelated aspect, disclosed is a method for anchoring soft tissue. The method includes constraining a self-expanding anchor device within a lumen of a constraining element. The self-expanding anchor device includes a plurality of struts extending between a distal end region and a proximal end region of the anchor device and at least partially surrounding an internal volume of the anchor device. The device includes an attachment feature positioned near the distal end region of the anchor device, and a proximal opening into the internal volume. At least a portion of the plurality of struts is constrained by the constraining element and at least a portion of the distal end region extends beyond a distal edge of the constraining element. The method includes securing a material to the attachment feature and routing the material through the internal volume of the anchor device. The method includes penetrating a bone surface with the distal end region of the self-expanding anchor device until the distal edge of the constraining element abuts the bone surface. The method includes sliding an advancing element relative to the constraining element urging the anchor device beyond the distal end of the constraining element into a subcortical location of the bone surface. The method includes passively expanding the plurality of struts within the subcortical location. The material can include suture or cable. The material can be secured to soft tissue.

In an interrelated aspect, disclosed is an implantable fixation device formed at least in part of temperature affected shape set material that transitions from a geometrically confined configuration to an expanded configuration. The device is constrained to the confined configuration at ambient storage temperatures by a removable element. The removable element is removed after the temperature affected shape set material is chilled immediately prior to delivery into the body.

The removable element can be generally tubular. A secondary confinement tubing or ring element can be positioned over a distal segment of the removable element during storage. The secondary confinement tubing can be removed after chilling the self-expanding device immediately or shortly prior to deployment within the bone. The implantable fixation device can be an implantable soft tissue fixation device. The proximal end region can further include a plurality of serrations extending from a proximal edge of the device. A proximal end region of the device can further include a plurality of serrations extending from a proximal edge of the device. The plurality of serrations can capture soft tissue between the proximal end region of the device and at least a portion of bone through which the device is implanted.

In an interrelated aspect, disclosed is a suture or soft tissue anchor assembly having an anchor device with a plurality of self-expanding stays extending between a proximal end region and a distal terminus and surrounding an internal volume. A soft tissue material extends through the internal volume and loops over the distal terminus of the anchor device. The soft tissue material is approximated or secured to the anchor device at the distal terminus such that tension applied on the soft tissue material causes a force that foreshortens the length and expands the diameter of the anchor device.

The proximal end region of the device can further include a plurality of serrations extending proximally from a proximal edge of the device. The plurality of serrations can capture the soft tissue material between the proximal end region of the device and at least a portion of bone through which the device is implanted. The internal volume can extend an entire length of the anchor device from the proximal end region through the distal end region. The assembly can further include a cap covering an outer wall at the distal end region of the device. The cap can include a surface edge that is broader than a bare edge of the outer wall. The cap can be configured to support and redirect the soft tissue material looped over the distal terminus. The cap can be formed of a material that is softer than the material of the outer wall. The cap can be a polymeric material. The cap can further include one or more ridged surfaces. The one or more ridged surfaces can be oriented in a direction that is transverse to a longitudinal axis of the internal volume through which the soft tissue material extends. The one or more ridged surfaces can be configured to increase surface friction between the soft tissue material and the device to reduce soft tissue migration relative to the distal terminus.

In an interrelated aspect, disclosed is an anchor device for attaching materials within bone having a body having a distal end region, a proximal end region, and a tapered sidewall between the distal end region and the proximal end region. The body defines a bore extending along the body from the distal end region to the proximal end region. The anchor device includes a soft-tissue directing feature at the distal end region of the body configured to direct a soft-tissue that extends through the bore in a first direction to a second direction at an angle to the first direction. The body of the anchor device is contracted from an initial configuration to a delivery configuration in which an external diameter of at least a portion of the body is reduced, and return to the initial configuration upon release.

The anchor device can be contracted radially of the body. The anchor device can be contracted in an arcuate manner. At least the portion of the body can have a c-shaped cross-section with an interrupted outer wall portion. The interrupted outer wall portion can have an interruption that is dimensioned to control a maximum reduction in the diameter of the body. The anchor device can further include a soft-tissue confinement feature at the distal end of the body and adjacent to the soft-tissue directing feature. The soft-tissue confinement feature can be configured to confine the soft-tissue that passes through the bore of the body and directed by the soft-tissue directing feature. The soft-tissue confinement feature can include a pair of opposed prongs extending at the distal end of the body. The anchor device can include a soft-tissue compression collar at the proximal end portion of the body. The anchor device can include a retention shelf near the proximal end portion of the body. The tapered sidewall can taper from the retention shelf to the distal end portion. The retention shelf can include the portion of the body that is reduced when contracted. The anchor device can include a soft tissue-compression collar at the proximal end portion of the body, adjacent the retention shelf. The retention shelf can further include one or more ridging surfaces. The one or more ridging surfaces can be arcuate. The soft-tissue directing feature can include one or more ridging surfaces to increase friction with the soft-tissue. The body can be monolithic and can have a substantially conical shape. The tapered sidewall can include one or more scalloped features. The body of the anchor device can be configured to flex generally along a longitudinal axis of the body.

In an interrelated aspect, disclosed is a system for securing tendon to bone having a pliable and elastic body having a transverse cross-sectional geometry that is generally arcuate-shaped forming a concave and a convex surface to the body. The body has a generally orthogonally-oriented long axis extending between a distal end and a proximal end. An arcuate length of the transverse cross-section near the distal end is shorter than an arcuate length of the transverse cross-section near the proximal end. When in use within a bored defect within a bone, the distal end of the body is positioned within a depth of the medullary cavity of the bone and the proximal end of the body is positioned within the bone near a cortical surface of the bored defect. The system includes a tendon positioned along the long axis of the body coursing along both the concave and the convex surfaces of the body such that the course of the tendon is sharply redirected by wrapping around the distal end of the body while a segment of a detached end of the tendon is compressed between the convex surface of the body near the proximal end and the cortical surface of the bored defect.

The body can have a generally hemi-frusto-conical geometry. The anchor system can further include distally projecting elements forming a lateral confining element within which the tendon courses around the distal end of the body. The arcuate length of the proximal end of the body can be greater than a diameter of the bored defect. A transverse arcuate wall thickness of the body can be non-uniform. The transverse arcuate wall thickness can be greater near a margin than in a central region. The body can be composed of elastomeric material such as PolyEtherEtherKetone (PEEK) polymer or Nitinol alloy.

In an interrelated aspect, disclosed is a pliable and elastic device for securing tendon to bone that is configured to be driven into a cortical cancellous bore initially resulting in radial contraction and arcuate hinging of the device through a region of the cortical bone with subsequent relaxation of the device in a region of subcortical bone.

A diameter of a portion of the proximal subcortical arcuate segment of the device can be greater than a diameter of the cortical bore through which the device is inserted. The device can be composed of elastomeric material such as a PEEK polymer or Nitinol alloy.

In some variations, one or more of the following can optionally be included in any feasible combination in the above methods, apparatus, devices, and systems. More details of the devices, systems and methods are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally speaking, the figures are not to scale in absolute terms or comparatively but are intended to be illustrative. Also, relative placement of features and elements may be modified for the purpose of illustrative clarity.

FIG. 1 shows a perspective view of an implementation of an anchor device in an unexpanded state.

FIG. 2 shows a perspective, exploded view of the anchor device of FIG. 1 in an expanded state.

FIG. 3 shows a perspective, exploded partial view of the anchor device of FIG. 1 without struts.

FIG. 4A shows a perspective view of an implementation of an anchor device in a constrained configuration.

FIG. 4B shows a perspective view of the anchor device of FIG. 4A and an exploded view of an implementation of a deployment tool.

FIG. 4C shows a perspective view of the anchor device and deployment tool of FIG. 4B with an associated soft tissue and relative to a bone.

FIG. 4D shows a perspective view of the anchor device of FIG. 4A with an associated soft tissue.

FIG. 4E shows a perspective view of the anchor device of FIG. 4D with an associated soft tissue and in an expanded state.

FIGS. 4F, 4G, and 4H show various views of the anchor device of FIG. 4E with an associated soft tissue in an expanded state and deployed within a bone channel.

FIG. 4I shows another implementation of an anchor device with an associated soft tissue in an expanded state and deployed within a bone channel.

FIG. 5 shows a perspective view of an implementation of a cleat element for tensioning sutures to the anchor device of FIG. 1.

FIG. 6 shows a perspective view of the cleat element in FIG. 5 held in a compact configuration with an associated suture in a confining element.

FIG. 7A shows an implementation of an anchor device in an expanded state and including a cleat element.

FIG. 7B shows an example suture pattern or formation for unidirectional suture tensioning.

FIG. 8 illustrates an implementation of an anchor device coupled to an implementation of an implant deployment tool in a constrained configuration.

FIG. 9 illustrates the anchor device of FIG. 8 advanced along a part of the implant deployment tool and in an unconstrained configuration freeing the anchor device to expand and become secured to bone material.

FIG. 10 illustrates the anchor device of FIG. 9 in an expanded configuration with suture extending from the anchor device and secured by a suture loop to soft tissue.

FIG. 11 illustrates a side view of an implementation of an anchor device;

FIG. 12 illustrates a side view of the anchor device of FIG. 11;

FIG. 13 illustrates a perspective view of the anchor device of FIG. 11;

FIG. 14 illustrates an opposite side view of the anchor device of FIG. 12;

FIG. 15 illustrates a perspective view of the anchor device of FIG. 11;

FIG. 16 illustrates a perspective view of the anchor device of FIG. 11 anchored into bone;

FIG. 17 illustrates a perspective, distal end view of the anchor device of FIG. 11 anchored into bone;

FIG. 18 illustrates a side view of the anchor device of FIG. 11 anchored into bone;

FIG. 19 illustrates a front view of another implementation of an anchor device;

FIG. 20 illustrates a rear view of the anchor device of FIG. 19;

FIG. 21 illustrates a front perspective view of the anchor device of FIG. 19;

FIG. 22 illustrates a rear perspective view of the anchor device of FIG. 19;

FIG. 23 illustrates a perspective view of the anchor device of FIG. 19 in use, following insertion;

FIG. 24 illustrates a side view of the anchor device of FIG. 19 in use, following insertion;

FIGS. 25-26 illustrate perspective views of an implementation of an anchor device;

FIG. 27 illustrates a view of the anchor device of FIGS. 25-26 posed superficial to a segment of soft tissue juxtaposed to a unicortical blind defect in a bone;

FIG. 28 illustrates the anchor device of FIG. 27 after being driven into the defect;

FIG. 29 is a top end view of the anchor device of FIG. 28 positioned within the defect;

FIG. 30 is a side, cut-away view of the anchor device of FIG. 28 positioned within the defect;

FIG. 31 is a cross-sectional view of an anchor device coupled to an insertion tool.

It is to be understood that implants described herein may include features not necessarily depicted in each figure.

### DETAILED DESCRIPTION

During at least some orthopedic surgical procedures, surgeons can attach or reattach soft tissue structures to bone via suture material. A suture anchor device can provide a way for anchoring to the bone, such as cortical or cortical cancellous fixation. Fixation can be provided by an implant having interference fit, (e.g., with the tendon compressed between the implant and the bone) or a thread, suture or cable attachment form. Alternatively, fixation of the implant can be achieved by radial and/or implant hinge expansion within the subcortical cancellous bone. It is also proposed that a combination of radial/hinge expansion and tendon or ligament compression can be employed, as is utilized in some implementations of this invention. The expansion can occur beyond the cortical defect through which the implant was inserted such that pull-out of the implant is resisted.

Described herein are devices, systems and methods of use to provide a rapid, easy and reliable way to anchor sutures or soft tissues such as tendon, ligament or joint capsule to bone. Implementations of the anchor devices described herein are simple to deploy in that they are either constricted on insertion, self-expanding, or both, and do not require active expansion. In a preferred implementation of the tendon anchor that does not utilize suture fixation, the anchor devices described herein can be constricted during deployment, and once deployed, return to an initial size without additional expansion. The anchor devices described herein allow for better vascular growth and more surface fixation, than other marketed devices that are less porous or shield the attached soft tissue from adjacent bone. The anchor devices described herein minimize the strangulation of the soft tissue to be affixed or the trapping of the soft tissue between rigid parts of the anchor device or the bone channel. The anchor devices described herein can saddle and redirect the soft tissue to be affixed within the interior volume of the device while still allowing for direct intimate contact of the soft tissue with the bone to which it is being affixed thereby promoting bone and soft-tissue attachment to the secured tissue.

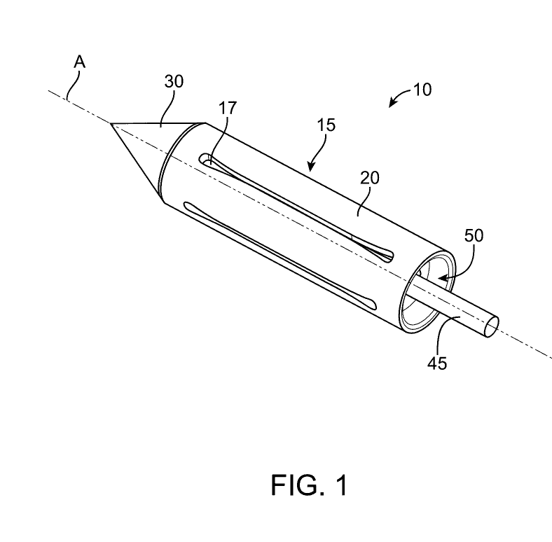

FIG. 1 shows an implementation of an anchor device 10. The anchor device 10 can be configured to anchor suture to bone. The anchor device 10 can include a cylindrical outer body 15 having a plurality of slots 17 forming a plurality of stays or struts 20. The anchor device 10 can include two or more longitudinally extending slots 17 along its long axis A and on its tubular outer body 15 that can define the edges or margins of the deployable struts 20. The struts 20 can be located along the central portion of the generally tubular portion of the anchor device 10. The tubular outer body 15 can be coupled to an inner element 25 having a piercing distal tip 30. The distal tip 30 can be pointed or conically tapered to facilitate penetration of bone. The distal tip 30 can be configured with trephine or fluted geometry to facilitate penetration of bone. It should be appreciated that the distal tip 30 need not be integrated with the inner element 25 and can be coupled to a distal end region of the outer body 15 according to other configuration.

In some implementations, the anchor device 10 can have a long axis A, defining a generally tubular or cylindrical body geometry to the anchor device 10 immediately prior to insertion and delivery. The long axis A can have a proximal end that can be superficial in location and a distal end that can be deep in location with respect to the patient's bone surface or cortex. The proximal end of the anchor device 10 can be tapped such as with or through a deployment tool to force the sharp distal tip 30 through bone material. The proximal end of the anchor device 10 can also be pushed to urge the anchor device 10 through a pre-drilled hole.

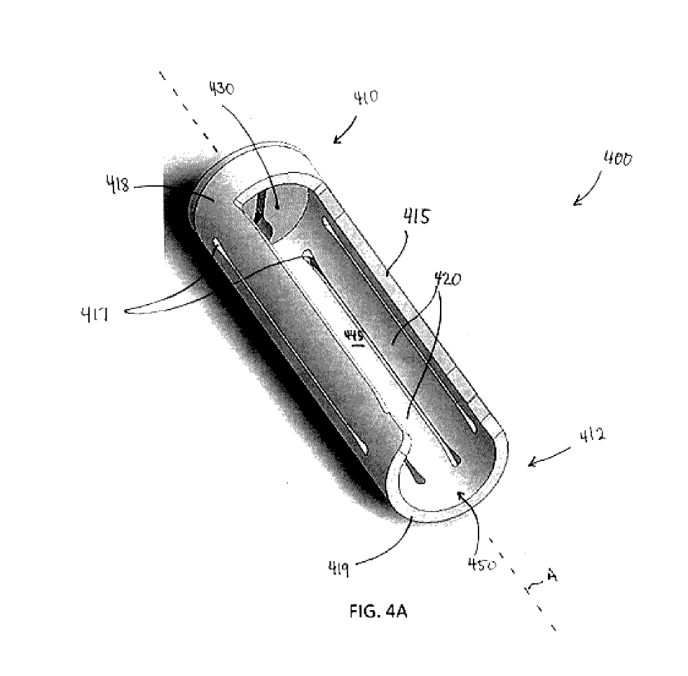

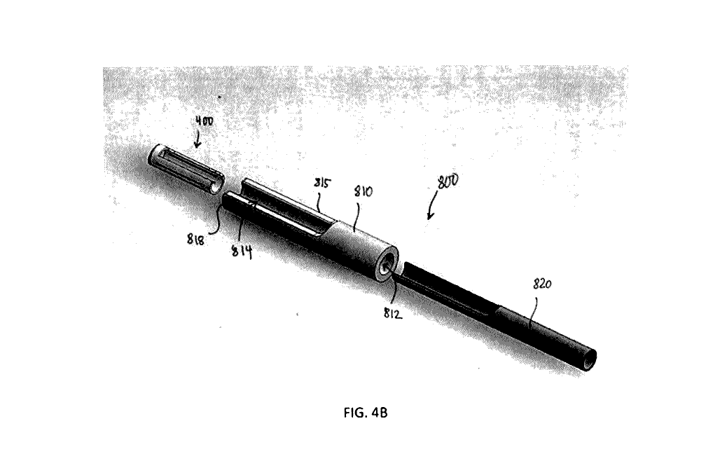

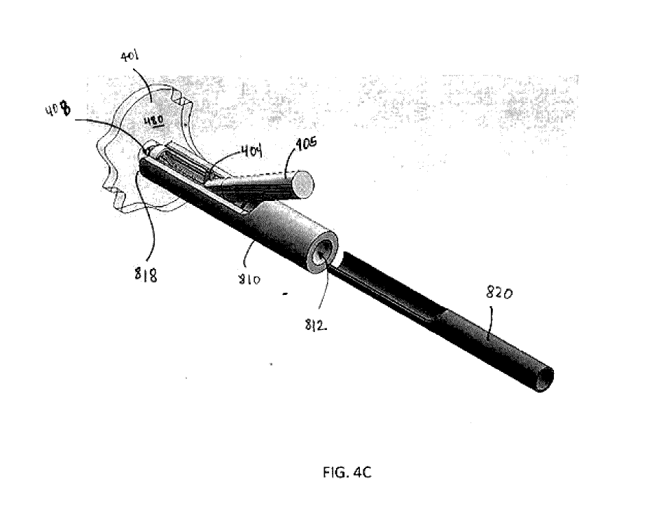

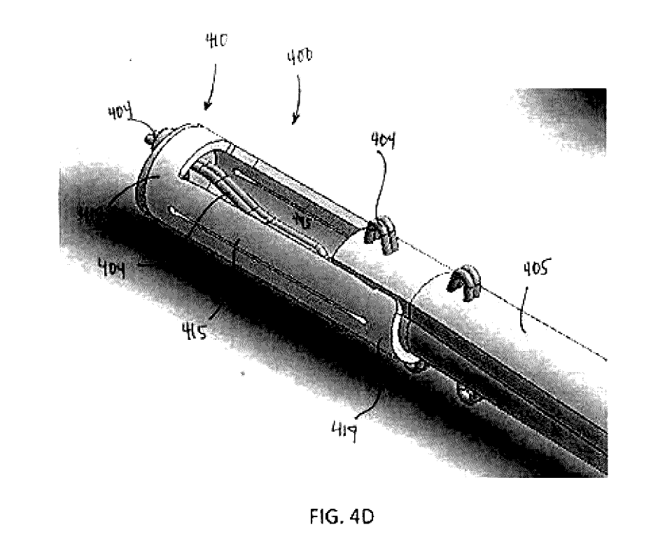

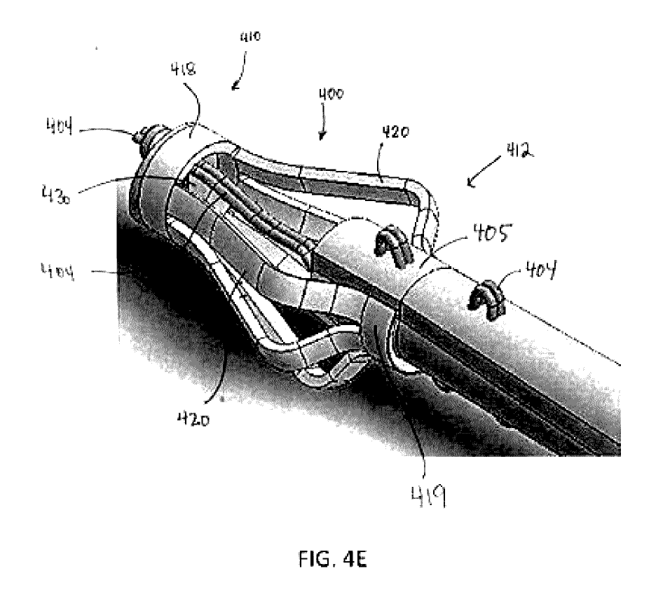

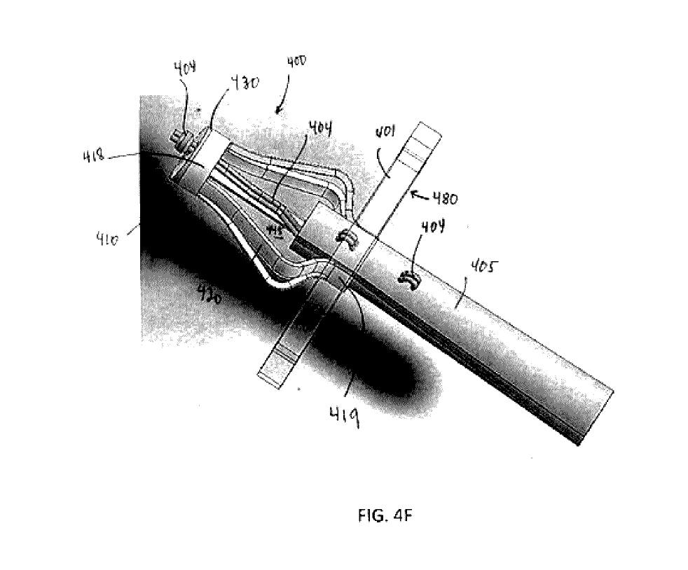

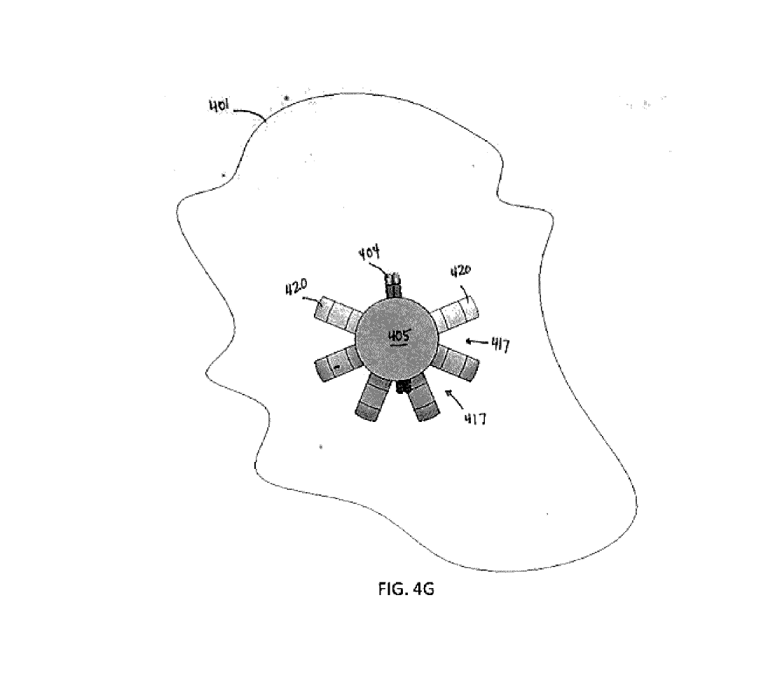

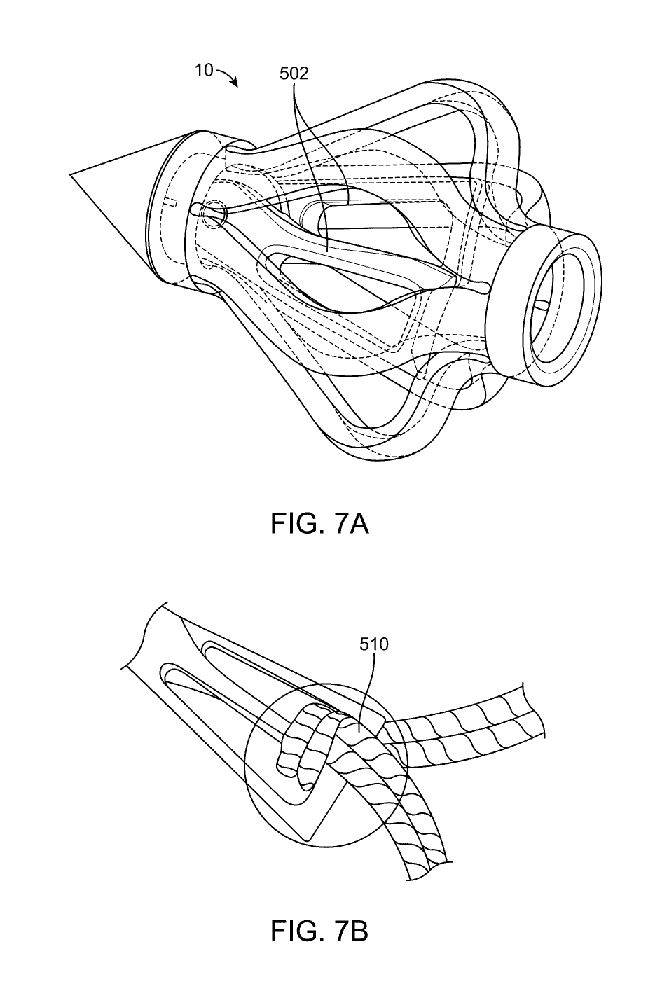

In some implementations, the outer body 15 of the anchor device 10 can be self-expanding. The outer body 15 can be fabricated from super-elastic shape memory metal, such as Nitinol. Prior to deployment, the struts 20 of the outer body 15 can be constrained by an implant deployment tool (for example, like the tool 800 shown in FIG. 8) such that the plurality of struts 20 are constrained into a first configuration having a reduced diameter geometry, for example prior to delivery into bone. The plurality of struts 20 can also be configured to expand to a second configuration having enlarged diameter geometry upon removal of the constraint after delivery in the bone. The deployment tool 800 can include a constraining element 810 such as an external tube having a distal wall slot 814 (see FIG. 4B) to accommodate suture delivery and suture deployment tensioning as will be described in more detail below. Upon deployment, the superelastic shape memory struts 20 can self-expand in a radially disposed manner while simultaneously foreshortening along the overall longitudinal axis A of the anchor device 10. The anchor device 10 can radially expand within the subcortical cancellous bone to a dimension or dimensions that exceed the dimension of the generally round cortical defect through which the anchor device 10 was introduced. In the unexpanded state, the anchor devices described herein can be between about 3 mm-8 mm in diameter and can be between about 10 mm-30 mm in length. In some implementations, the anchor device is approximately 6 mm in diameter and 20 mm in length. It should be appreciated that the anchor devices described herein can be deployed using passive or active deployment or a combination of the two. For example, the anchor devices described herein can undergo initial passive deployment for provisional fixation in the bone channel and then active tensioning for full and final expansion.

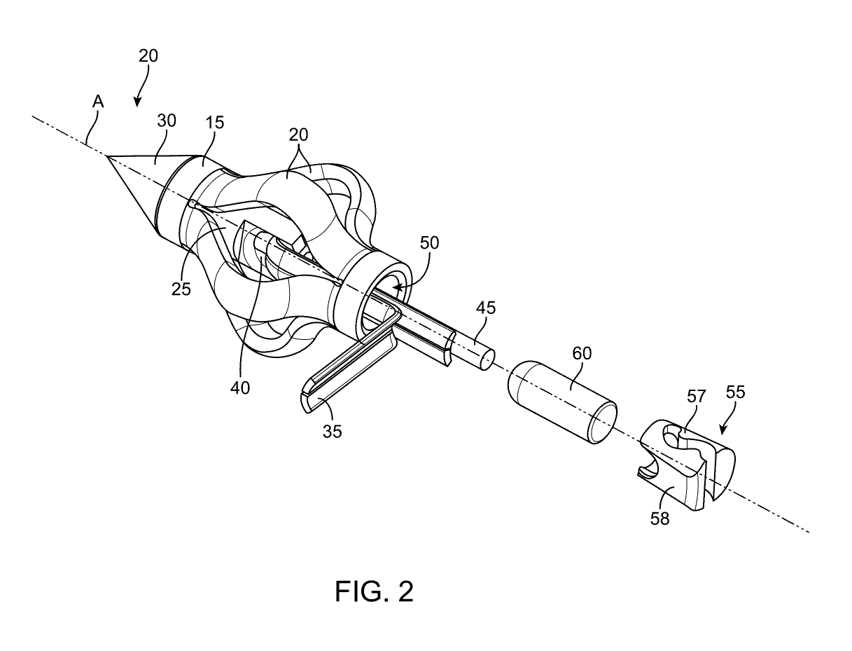

As shown in FIG. 2, suture strands 35 can be affixed to body tissue (primarily soft tissue, such as ligament, tendon, or joint capsule) and advanced through the proximal aspect of the distal wall slot 814 of the constraining element 810 of the deployment tool 800 (see, for example, FIG. 4B-4C) and through the proximal aperture 50 of the device's tubular body 15 prior to placing the anchor device 10 within the bone. The suture strands 35 can be advanced distally along the longitudinal axis A of the anchor device 10 to a distal suture attachment feature 40 of the device. The attachment feature 40 can be a pulley, cleat or other element having similar configuration and/or function to progressively tension the sutures and approximate the attached soft tissue element to the proximal aspect of the device. The attachment feature 40 can facilitate the redirection of the suture material 35 providing for the suture ends to be delivered again along the longitudinal axis A back towards a proximal end of the anchor device 10 and back out through the proximal aperture 50 and, in some implementations, into a handle mechanism of the deployment tool 800.

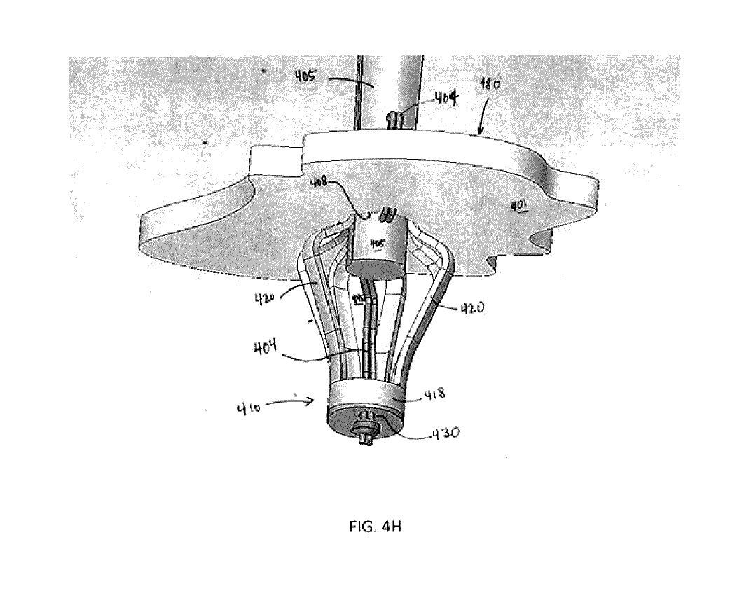

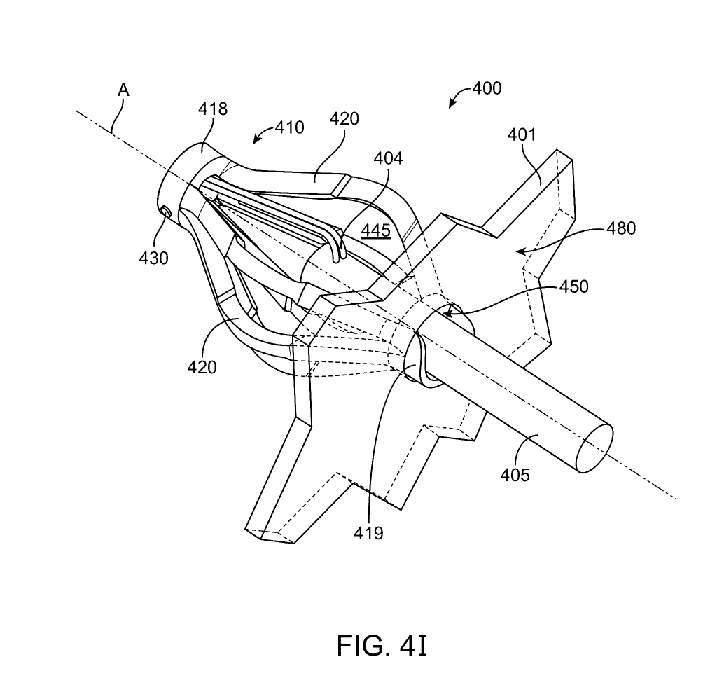

Other configurations of the attachment feature 40 are considered herein, such as those described in FIGS. 5 and 6. For example, FIG. 5 shows an interrelated implementation of an attachment feature 500 that can be used to progressively tension and lock suture to the anchor device 10. The attachment feature 500 can include a pair of suture anchoring elements 502 coupled together, for example by a pin 504, forming a collapsible cleat device. The pin 504 can couple the pair of suture anchor elements 502 together such that they can be tethered or hinged or articulate relative to one another between a generally aligned position to a generally splayed configuration. The pin 504 can be associated with a distal aspect of the anchor device 10, for example attached to or integrated with the body of the anchor device 10, such that the attachment feature 500 is otherwise affixed to the anchor device 10.

Additionally, each of the suture anchor elements 502 can include at least one aperture 506 that can allow suture 510 or a pliable suture passing component, to pass through. This can allow for a variety of methods of securing suture 510 to the suture anchor elements 502 and the anchor device 10. For example, the suture 510 can be fixed to the attachment feature 500 by passing at least a part of the suture 510 through the proximal aperture 50 of the anchor device 10 and into the interior cavity (including following deployment of the device). In addition, at least a part of the suture 510 can be routed through the aperture 506 of at least one of each of the suture anchor elements 502 (see FIG. 5). The suture 510 can be routed in such a manner as to position a part of the trailing portion of the suture (i.e. those parts of the suture 510 closer to the soft tissue that is being approximated and anchored to the bone), underneath a portion of the leading portion of the suture (i.e. the portion of the suture that is closer to the end of the suture that is being pulled in tension), such that the tensioned suture will resist loosening due to friction resulting from one portion of the suture compressing another portion of the same suture. This effectively results in a unidirectional tensioning or cleating effect.

The suture 510 routed through the proximal aperture 50 of the anchor device 10 (as seen in FIG. 1), and then through the attachment feature 500 apertures 506 of one or both suture anchor elements 502 (seen in FIGS. 5-6). The suture 510 can also include at least one circumferential wrap around a common post 512 of the distal suture attachment feature 500. The common post 512 can include adjacent or opposing post elements from each of the suture anchor elements 502. Once the suture 510 has been wrapped around the common post 512, the suture 510 can be routed back out of the proximal aperture 50 of the device 10. The routing of suture material in such a manner as to have the trailing portions of the suture material (i.e., the part of the suture material that is closest to or attached to the soft tissue) overlap those portions of the suture material that are being actively tensioned (i.e., the part of the suture material that has been routed back through the proximal aperture) can assist in creating a cleat-like unidirectional tensioning of the suture. The cleat mechanism of the attachment feature 500 can allow tensioning of the suture 510 while preventing subsequent loosening of tension placed on the suture 510 associated with the suture anchor elements 502 and the soft tissue to which the suture 510 is attached.

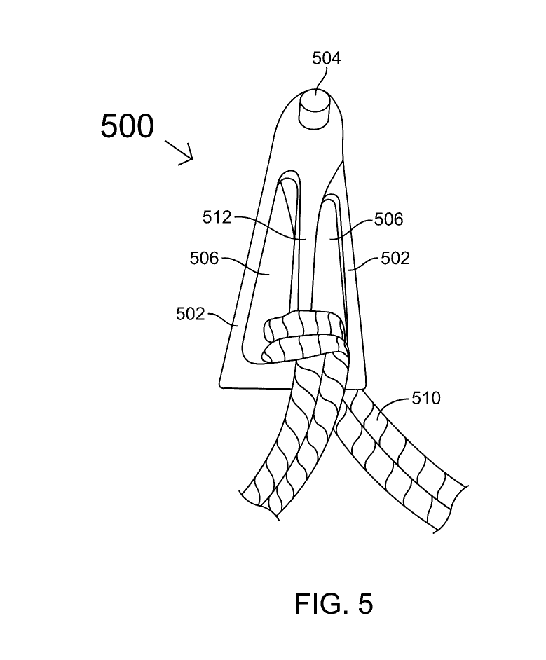

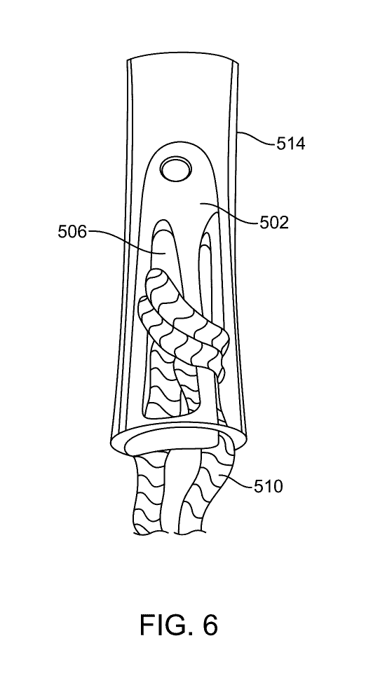

FIG. 7A shows an interrelated implementation of the anchor device 10 in an expanded configuration and having a pair of suture anchor elements 502 (suture not shown). As described above, the suture anchor elements 502 can provide tensioning of suture, such as unidirectional suture tensioning, which can assist in securing the positioning of tissue within a patient's body. FIG. 7B shows an example suture pattern or formation for unidirectional suture tensioning. In addition, the unidirectional suture tensioning formation can be formed using the suture anchor elements for tensioning suture and positioning tissue associated with the suture.

FIG. 8 illustrates an implementation of an anchor device 10 coupled to an implant deployment tool 800 in the constrained configuration. The implant deployment tool 800 can include a variety of features for assisting in positioning and deploying the anchor device 10, as well as for manipulating (i.e., cutting, tensioning, positioning, etc.) suture associated with the anchor device 10. As shown in FIG. 8, the implant deployment tool 800 can include a constraining element 810 which can assist in restraining the anchor device 10 and preventing the anchor device 10 from expanding, at least until desired. In addition, the implant deployment tool 800 can include an advancing element 820 which can assist with advancing and deploying the anchor device 10, as well as cutting the suture in order to remove excess suture.

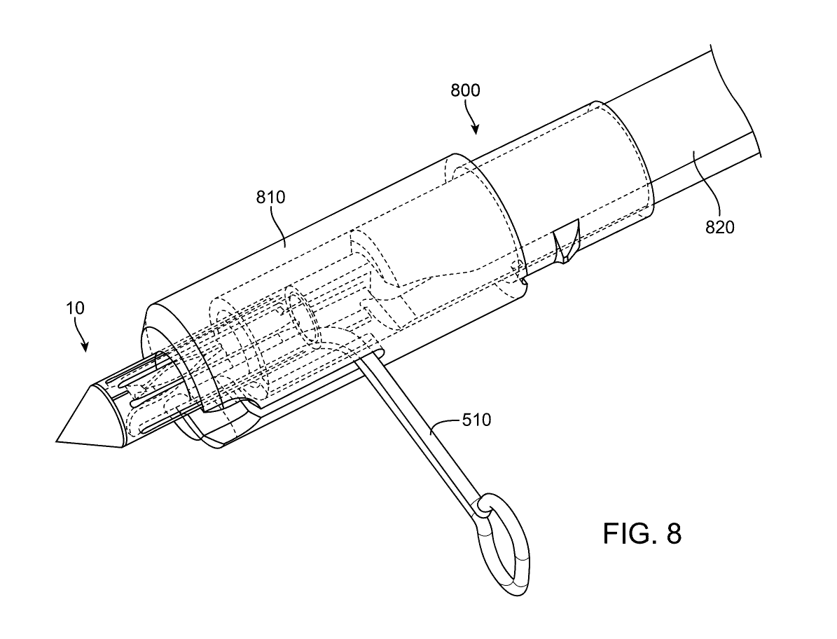

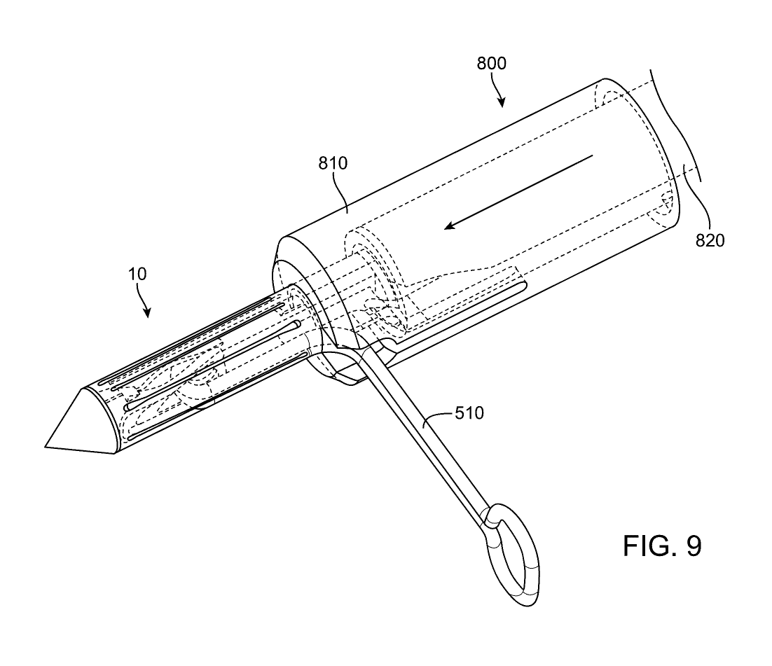

As shown in FIGS. 8, 9, and 10, suture 510 can be secured within the anchor device 10, such as secured to the suture anchor elements 502 as described above, and can extend from the anchor device 10 and implant deployment tool 800 in order to attach to tissue. The suture 510 can include a feature, such as a suture passing loop 830, for routing suture attached to soft tissue 835 as shown in FIG. 10. The advancing element 820 of the implant deployment tool 800 can be advanced or moved in order to allow the anchor device 10 to deploy (such as from the constraining element 810) and expand, as shown in FIG. 10, which can allow the anchor device 10 to become securely implanted in bone material and assist with securing tissue associated with the suture 510.

The attachment feature 500 of the device can be surrounded by the slotted tubular body 514 (see FIG. 6) having the plurality of struts in a confined state prior to deployment (see FIGS. 6 and 8) and expansion (see FIG. 10). Prior to deployment, the struts 20 of the device 10 can be contained or confined within a rigid, generally or nearly circumferential element, such as the constraining element 810, which can maintain the suture anchor elements 502 in a fully overlapped position (as shown in FIG. 6). The confining element, such as the constraining element 810, as well as the tubular body 514 can allow the attachment feature 500, including the pair of suture anchor elements 502, to be initially implanted and positioned in a confined configuration, such as in an overlapped configuration forming a reduced combined width while still accommodating the positioning of suture or suture passing material through the apertures 506. In addition, the constraining element 810 can allow the suture anchor elements 502 to hinge into an expanded position, such as pivoting relative to one another, upon removal or decoupling of the constraining element 810 allowing the struts 20 to expand, as shown in FIGS. 2 and 7A. Once the constraining element 810 has been removed or decoupled from the attachment feature 500, including the suture anchor elements 502, at least the attachment feature 500 can expand. The suture 510 that is looped through the apertures 506 of the suture anchor elements 502 and around the common post 512 can then be pulled in tension, which can cause the splaying of the suture anchor elements 502 within the expanded anchor device 10.

In addition, a loop on the suture 510 can be used to pull suture material or cable 830 that has been previously associated with soft tissue (e.g. tendon, biceps tendon, posterior tibial tendon, rotator cuff), through the suture anchor device along its routed path in the deployed or expanded position of the implant. The soft tissue structure can be pulled through the suture anchor device once the anchor has been inserted into the bone and deployed into an unconstrained configuration (as shown in FIG. 10). In addition, further tensioning of the suture 510 can approximate the soft tissue to which the suture 510 or suture material, such as a suture loop as illustrated in FIG. 10, is attached or associated with and hold the approximated soft tissue near or adjacent to the anchor device 10. Additionally, the cleat mechanism formed by the suture 510 and the suture anchor elements 502, acting effectively as a cleat, can assist in maintaining the suture in tension.

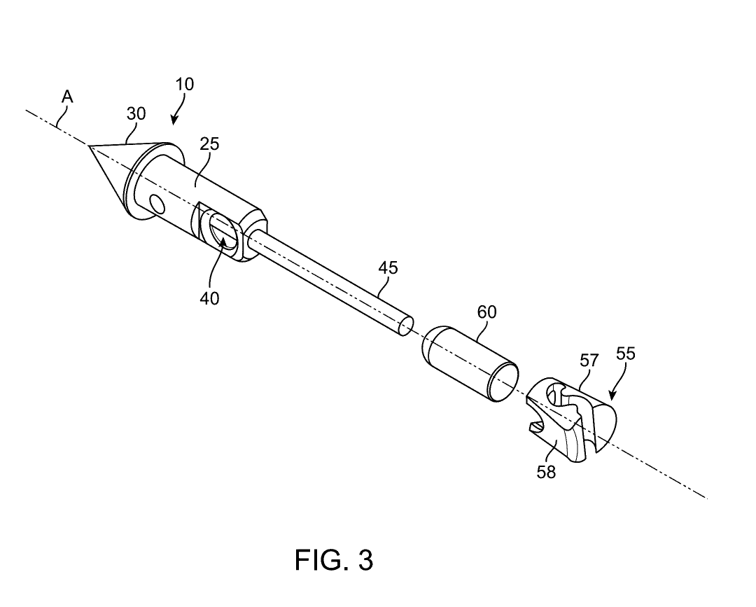

As shown in FIG. 3, the anchor device 10 can also include a tensioning element 45. The tensioning element 45 can function similar or identical to the suture loop 510, as shown in FIG. 10. The tensioning element 45 can be routed around a distal pulley or post from a fixed location, such as an attachment element at the distal end region of the anchor device 10, such as near the distal end region of the inner body 25 or the tubular body 15 of the anchor device 10, and returning proximally through the interior volume along the central longitudinal axis A of the anchor device 10 and through the proximal aperture 50 of the tubular body 15 of the anchor device 10. The central tensioning element 45 can be a cable or suture material, such as ultrahigh molecular weight polypropylene fiber cable, Dacron fiber cable, or a combination of synthetic implantable fibers. The tensioning element 45 can approximate the distal and proximal ends of the anchor device 10 as well as approximate the soft tissue to the bone within which the implant was deployed. The central tensioning element 45 can extend into a handle mechanism of the deployment tool. The proximal aspect of the central tensioning element 45 can extend within the deployment tool to a tensioning mechanism. Such a tensioning mechanism can include an active ratcheting element or passive spring, which when deployed can provide loading of the tensioning element 45, foreshortening of the anchor device 10 as the proximal end region and the distal end region are brought towards each other as radial expansion of the device's perimeter struts 20 occurs. It should be appreciated that the device 10 can be self-expanding, manually expanding, as well as a combination of self-expanding and manually expanding. For example, the anchor device can self-expand to a degree upon release of a constraint on the struts 20 and then manually expanded using tensioning mechanism such as the tensioning element 45 to cinch the ends toward one another to achieve a fully expanded maximal diameter.

With the perimeter struts 20 fully deployed (see FIG. 2), a mechanism within the deployment tool 800 can provide for tensioning of the suture strands 35 and approximation of tissue, such as soft tissue like a tendon, to which the suture strands 35 are affixed toward the proximal aperture 50 of the implant's tubular body 15. Once sufficient tensioning of the suture strands 35 has been deemed to have occurred, a peripherally hinged crimping element 55 can be advanced distally from the deployment tool along the longitudinal axis A of the anchor device 10 confining and ultimately trapping the suture strands 35 near and/or within the proximal aperture 50 of the tubular element 15. In some implementations, the crimping element 55 can have a generally conical external geometry, initially hinged open to accommodate the tensioning of the central tensioning element 45 and the suture strands 35, prior to crimping. In addition, as the crimping element 55 can be urged within the proximal aperture 50 of the tubular element 15, the interaction of the internal bore of the tubular element 15 with the external geometry of the crimping element 55 can result in the crimping element 55 hinging closed to trap and fix the tension of the suture stands 35.

In some implementations, the opposing internal surface features 57, 58 of the crimping element 55 can interdigitate with closure, providing for optimized friction lock of the suture strands 35 and central tensioning element 45 within. The surface features 57, 58 can additionally include opposing sharp proximal edges that either meet or overlap in a scissoring manner, resulting in division of the suture strands 35 and central tensioning element 45 at a tip of the crimping element 55 and most proximal aspect of the tubular body 15. The internal bore of the proximal aspect of the tubular body 15 of the anchor device 10 can have a conical geometry that can match the geometry of the external surface of the crimping element 55 in the crimped configuration. The crimping element 55 can also include a side slot for capturing the suture strands 35 within the crimping element 55 from the side.

In an implementation of deployment, a suture strand(s) 35 can be placed through a tissue or other material that is intended to be approximated to bone. The “free” suture ends 35 can be passed by way of suture passers (such as by wire cable or synthetic cable coursing along the intended course of the sutures within the device) that are pulled or tensioned along with the attached free ends of the suture 35 within the device deployment tool. With the sutures 35 secured to the device deployment tool 800, the pointed tip 30 of the anchor device 10, which can extend distally beyond the distal end of the constraining element, can be delivered along a soft tissue path that minimizes the potential for a soft tissue bridge (i.e. superficial soft tissue that is trapped between the tensioned suture strands and the bone). This can be accomplished with a variety of strategies, such as using an introductory cannula or by placing tension of the sutures 35 with one of the surgeon's hands and then sliding the delivery shaft and anchor device 10 immediately adjacent and along the axis of the tensioned suture strands 35 with the other hand.

Once the anchor device 10 is delivered into close proximity to the bone's cortical surface to which the suture strands 35 are intended to be approximated, the sharp distal tip 30; which might include a trocar geometry and/or very sharp tip, can be tapped through the cortex or pushed through a pre-drilled hole. The anchor device 10 and device deployment tool 800 can be advanced until the distal edge 818 of a constraining element 810, which can have a larger diameter than the cortical defect or channel through which the distal tip 30 has been advanced, is positioned up against the outer cortex of the bone (see, for example, FIG. 4C). The constraining element, such as the constraining element 810 shown in FIG. 4C or FIG. 8, can thus serve as a shoulder stop and stabilizing feature for the device deployment tool. A tubular deployment element, such as the advancing element 820 shown in FIGS. 4C and 8, can be advanced within the internal bore 812 of the constraining element 810, pushing on the proximal surface edge of the tubular outer body 15 of the anchor device 10, advancing the anchor device 10 within the bone such that the proximal edge of the proximal aperture 50 is generally located at or near the external cortical surface of the bone. This deployment can be accomplished by a push-pull mechanism or relative linear translation of the advancing element 820 relative to the constraining element 810 with reactive forces transmitted to the cortical surface of the bone by the shoulder restraining feature of the distal edge 818 of the constraining element 810. This can reduce the risk of unintended fracture or cracking of the cortical bone adjacent to the introductory cortical bore. Further, this can avoid the need to restrain the deployment forces imparted by the deployment mechanism 800.

Once delivered within the subcortical location, the anchor device 10 may be radially expanded via a passive process mediated or effected by the shape memory properties of the superelastic metal alloy of the struts 20. This can be followed by active tensioning of the suture leads until the suture is optimally tensioned and the attached tissue is sufficiently approximated to the devices cortical entry location. Tensioning of the suture leads can be accomplished with a variety of mechanisms, including opposing cam configured cleats or a ratcheting reel mechanism. In an implementation, the tensioning of the suture 510 and radial expansion of the struts 20 is accomplished via the unidirectional tensioning feature of the dual aperture and common post cleat elements, such as described above in reference to FIG. 5.

A crimping element 55 (shown in FIGS. 2 and 3) can then be advanced over or onto the tensioned central tensioning element 45 and the tensioned suture strands 35, until they are restrained from subsequent displacement. The crimping element 55 can include an element within which the suture strands 35 can be trapped by progressive approximation of opposing walls within the proximal aperture 50 of the anchor device 10. According to another implementation for securing the sutures and tensioning element(s), a tapered interference pin 60 can be delivered within the proximal opening or aperture 50 of the anchor device 10. The pin 60 can have sufficient cross-sectional area and length to provide for an interference fit with the surrounding suture 35 and tensioning element(s) 45.

A cutting tool or cutting feature can be situated at the upper end of the crimping element 55 can be used subsequent to the crimping or trapping of the tensioned suture 35 and tensioning element(s) 45 to cut the cable elements of the suture and tensioning element(s). The device delivery mechanism can be separated due to suture and central tensioning amputation from the delivered, deployed device, and approximated tissue. In an implementation, the suture 510 may be cut via a rotating blade element within a deployment tool's shaft, which can also be in close proximity to the proximal aperture 50 of the anchor device 10.

FIGS. 4A-4I show interrelated implementations of an anchor device 400 and a deployment tool 800. The anchor device 400 can include a body 415 having a plurality of slots 417 forming a plurality of stays or struts 420 at least partially surrounding an interior volume 445 of the body 415 (see FIG. 4A). The anchor device 400 can include two or more longitudinally extending slots 417 along its long axis A from a distal end region 410 to a proximal end region 412 on its body 415 that can define the edges or margins of the deployable struts 420. The anchor device 400 can employ similar radially expanding features to secure the anchor device 400 with the medullary cavity of the bone as described above.

The anchor devices described herein can be deployed using passive, self-expanding deployment and include a pre-deployment confined configuration. In some implementations, the confined or constrained configuration can be at least in part maintained by a circumferential ring or tubular element. While the suture anchor device is in a deployed state, the circumferential ring element can provide a suture abrasion protective function resulting from one or more of a variety of surface features and deployment positions (i.e., located between the suture material and the bone).

It should be appreciated that the anchor devices described herein can be deployed using passive or active deployment or a combination of the two. In some implementations, the plurality of struts 420 passively transition from a constrained, delivery configuration that is radially contracted and axially elongated to a relaxed, deployment configuration that is radially expanded and axially shortened. The anchor device 400 can include a self-expanding super-elastic shape set material, such as nitinol, that prior to deployment is maintain in a constrained configuration having a reduced diameter along a segment of its length that passively radially expands with deployment within the internal confines or medullary cavity of the bone. The anchor device 400 can rely solely upon the properties of the shape memory, super-elastic material (e.g. nitinol) to spontaneously revert (once unconfined) to a radially enlarged configuration with deployment. The anchor device 400 described herein can undergo initial passive deployment for provisional fixation in the bone channel and then active tensioning for full and final expansion. The passively deployed expansion can be purely within the subcortical region of the bone.

In some implementations, the anchor device 400 can be configured to anchor soft tissues 405 (see FIG. 4C) such as tendon or other tissue to or within a rigid material such as bone 401. The anchor device 400 can be used to deliver and secure a generally cylindrical segment of a detached tendon's terminus within a closely confining bore or channel 408 of bone 401. Examples of detached tendon can include the origin of the proximal long head of the biceps, the insertion of the biceps tendon in the proximal radius, extensor carpi radialis, the infrapatellar tendon of the quadriceps muscle, the anterior tibialis, the Achilles tendon, and extensor tendons of the digits. These tendons are generally detached from bone that has a dense cortical shell and a relatively soft medullary cavity. It should be appreciated, however, that the anchor devices described herein can be used to anchor other tissues and/or materials to bone as well.

The anchor device 400 can position the soft tissue 405, such as a tendon to be affixed, within the interior volume 445 of the highly porous body (for example, by virtue of the plurality of slots 417 and struts 415) of the expanded device 400 while providing for intimate contact with the cortical cancellous bone 401. The tendon or soft tissue 405 to be affixed can be secured to the anchor device 400 via a cable or suture 404 that is weaved through the soft tissue 405 and then tied or otherwise secured to the distal end region 410 of the anchor device 400 (see FIG. 4D). Thus, the tendon or soft tissue 405 can be tensioned separately from deployment of the anchor device 400 into the expanded configuration. A suture weave provides a safer way to secure the soft tissue 405 that is less likely to strangulate or prevent blood from circulating through the section of soft tissue 405 being secured which can necrose the soft tissue. Further, cradling the soft tissue 405 to be affixed within the highly porous architecture of the device 400 approximates the soft tissue 405 to the bone 401 in a manner that limits the compression of the soft tissue 405 against the cortical rim and affords abundant opportunity for vascularization of the soft tissue 405 as well as fibrous and boney attachments to the perimeter of the soft tissue 405. Further, the passive deployment of the anchor devices described herein upon insertion into bone such that expansion within the subcortical region occurs is less surgically challenging.

Loads imparted by tendon tensioning can be high and as such sufficient wall thickness and deployed rigidity are desired to overcome loads imparted by tendon tensioning and to prevent the anchor device 400 from collapsing through the small cortical defect through which it is inserted. The anchor device 10 described above may have substantially thinner device walls compared to anchor device 400 (or anchor device 600 described below) to facilitate active expansion using the central tensioning element.

A soft tissue 405, such as a tendon terminus, can initially be positioned and secured, through the proximal aperture 450 and into the bore or internal volume 445 of the body 415 (see FIG. 4D). In its confined and reduced diameter state, the body 415 forms a substantially tubular or cylindrical shape and the struts 410 surround the internal volume of the body 415. The soft tissue 405 can be secured to the anchor device 400 such as by an attachment feature 430 positioned within the internal volume 445 of the body 415 near a distal end region 410. The soft tissue 405 can be attached, for example, by weaving, tying or crimping or otherwise passing suture material 404 through the detached soft tissue 405. In an implementation, the suture material 404 attached to the soft tissue 405 is tied to an attachment feature 430 positioned within the internal volume 445 of the body 415 near the distal end region 410 of the implant 400, either before or after the implant 400 has been delivered with the soft tissue 405, into the bone 401.

The attachment feature 430 can be positioned within the internal volume 445 of the body 415 for example near the distal end region 410 (e.g. the end of the anchor device 400 that is first introduced through the bone 401) and can be configured to secure the tissue 405 to be attached to the bone 401 to the anchor device 400. The attachment feature 430 can include a post, slot, pulley, cleat, crimping element or other element as described herein to facilitate securing or coupling of materials such as a suture material and/or a soft tissue 405 to the anchor device 400. In some implementations, the attachment feature 430 can include a saddle shaped element to which at least two suture ends 404 can be passed around and subsequently knotted or crimped to secure the tendon 405 associated with the suture 404 within and/or to the anchor device 400 (see FIG. 4F). In other implementations, the attachment feature 430 can be a post extending transverse to the long axis of the device (see FIG. 4I) near the distal end region 410.

Once secured to the anchor device 400, the soft tissue 405 and the anchor device 400 can be delivered from the exterior surface 480 of the bone 401 through an appropriately sized and fashioned channel 408 or cortical defect (e.g. a drill hole sized to near the diameter of the tendon-device construct into the medullary cavity or internal canal of the bone 401) (see FIGS. 4C and 4F). A mechanical constraining element 810 of the deployment tool 800 can be positioned to surround at least a portion of the device 400 maintaining the struts 420 in a generally straight, reduced diameter configuration suitable for insertion through the channel 408. The constraining element 810 can be a relatively rigid tube or a ring-like structure. In some implementations, the constraining element 810 can be a tubular structure having an outer wall 815 surrounding an inner channel 812 and having a slot 814 extending through at least a portion of the outer wall 815 (see FIG. 4B). Separation of the constraining element 810 from the anchor device 400, permits the shape set super-elastic material of the anchor device 400 to spontaneously revert to its shape set deployed geometry having an enlarged diameter (see FIG. 4E). The enlarged diameter of the deployed configuration can be sufficiently larger than the bone channel 408 or cortical defect through which the anchor device 400 was introduced. This prevents and/or resists potential disengagement of the anchor device 400 and its attached soft tissue 405 from the bone 401, for example when placed under high loads during tendon tensioning and muscle contraction.

The struts 420 can be asymmetrically configured, such that when the device is deployed and the struts 420 expand radially from the long axis A of the anchor device 400, tensioning on the attached soft tissue 405 can result in off axis (i.e. tilting) displacement of the device, providing enhanced resistance to undesired displacement and “explantation” of the device and tendon from the bone's medullary cavity. In some implementations, the struts 417 can be shaped such that they have non-uniform wall thickness. For example, the struts 417 can be thinner near a central region and thicker near the distal and proximal ends. In other implementations, the struts 417 can have a reduced wall thickness where the struts couple to a ring-like structure that constrains the struts 417 and keeps the anchor device 400 in the reduced diameter configuration. The outer surface of the anchor device 400 can have a generally constant external diameter.

The anchor devices described herein, once implanted, can be put under a tensile load along the longitudinal axis A of the device. The tensile loads applied along the longitudinal axis A to the anchor device when in use can further approximate the distal end region to the proximal end region and result in further expansion of the struts away from the longitudinal axis A. Thus, the tensile load can act to further anchor the anchor device within the bone. In some implementations, suture or cable can be weaved through a detached tendon end that is being attached or repaired into the bone. The suture or cable can be tied or otherwise fixed to an attachment feature near or at the distal end of the device. As the tendon is placed in tension via muscle action or any other effect (e.g. elbow extension with the biceps tendon), the tension is transmitted to the distal end region of the device where the suture or cable material is attached. This tensile loading of the tendon and attached suture or cable maintains the anchor in the expanded configuration in which the distal end of the device is approximate to the proximal end of the device. The proximal end of the device is restrained in that the expanded struts are located immediately deep to the smaller diameter cortical defect. Thus, the device is kept compressed as a result against the deep surface of the cortical bone and the struts are in the deployed or expanded configuration.

The anchor devices described herein can also provide for limited contact with the surface features of the reattached soft tissue to optimize the biological repair process, for example, vascular and collagen repair within the bone to the tendon. In some implementations, the generally tubular geometry of the anchor devices described herein (at least in the constrained configuration) can be circumferentially disrupted along a segment of its proximal length while preserving circumferential continuity distally such that direct contact can occur between the soft tissues such as a tendon terminus and the adjacent tissues for optimal biological repair.

As best shown in FIG. 4A, the body 415 in its constrained configuration can be a generally cylindrical element having a plurality of slots 417 extending through the wall of the body 415 forming the plurality of struts 420. The plurality of slots 417 can be generally shorter in length than the overall length of the body 415 such that an outer wall 418 can be formed at the distal end region 410 of the body 415 and an outer wall 419 can be formed at the proximal end region 412 of the body 415. The outer wall 419 at the proximal end region 412 of the body 415 can form or define the proximal opening 450 into the internal volume 445 of the body 415. The outer wall 419 at the proximal end region 412 of the body 415 can be discontinuous forming a gap such that in cross-section it defines a generally c-shaped proximal opening 450 to the internal volume 445. Upon deployment of the anchor device 400, the discontinuous proximal outer wall 419 can be positioned near or within the bone channel 408 through which the anchor device 400 was delivered (see FIGS. 4E-4F). The gap or circumferential discontinuity in the proximal outer wall 419 of the otherwise cylindrical shape of the body 415 allows for at least a portion of the soft tissue 405 being delivered to be placed in direct intimate contact with the channel 408 or cortical defect in the bone 401 and medullary cavity of the bone, particularly the closely sized cortical defect, promoting tendon to bone healing. The gap in the proximal outer wall 419 can also provide for easier introduction of the tendon terminus 405 within the cavity or channel of the device and allow for asymmetric deployment/expansion of the device. Having the tendon or soft tissue 405 exposed along the circumference of the tendon-device construct and sizing the cortical defect 408 (e.g. drill hole) to provide for an intimate fit, can enhance not only the initial fixation but can also improve long term tendon biological fixation.

The devices described herein can be sterile packaged and confined in a delivery tube, with or without suture passers that can be employed to facilitate delivery of the suture strands (securely attached during surgery to the tendon) through the channel or aperture of the device and around the distal terminal post or saddle. A slot in the confining tubes distal end can accommodate the introduction of the tendon terminus. A small ring or grommet can be employed to maintain hoop strength of the confining delivery tube and serve to reduce abrasion after implantation of the tendon against a sharp external cortical edge.

A sizing guide can be used to determine the optimized size for the cortical defect (e.g. drill hole) needed to provide for intimate contact of the exposed tendon's surface with the cortical margin. The defect can be slightly undersized relative to the cross-section or diameter of the tendon-device construct such that with introduction, the compliance of the tendon can provide for annular constriction of the tendon within the cortical defect. This can be facilitated by tightly winding an implantable low friction monofilament suture material around the delivery tube and tendon secured to the device prior to delivery. The low friction monofilament material can temporarily constrict and confine the tendon to a reduced cross-sectional geometry and with deployment introduction remain superficial to the bone (allowing for its removal) allowing the tendon to relax back into a geometry of larger cross-sectional area within the medullary cavity.

In another implementation of deployment, a constraining tube element with or without a disruption of the circumferential continuity of the constraining element can be further surrounded by a secondary confinement element to reinforce the inner constraining tubing element in its ability to constrain the self-expanding device in its reduced diameter, constrained geometry. The constraining element can have a disruption of the circumferential continuity, as in a distal longitudinal slot feature (e.g. to accommodate suture introduction and delivery to the confined device), or a thin walled constraining tube that alone and at room temperature would be insufficient to constrain the outward expanding forces of the constrained self-expanding device. A secondary constraining ring or tube can be maintained around the inner constraining tubular element to reinforce the constraining effect. The distal tip of the device can be chilled prior to removing the secondary external ring or tube, immediately or shortly prior to deploying the self-expanding device.

The outward displacing forces of the shape set material within a thin walled and/or slotted tubing may exceed the circumferential restraining strength of the constraining tubing (i.e. hoop strain resulting in deformation of the tubing or splaying). An alternative accommodation can be to provide sufficient restraint at room temperature storage of inventory or above to avoid thicker walled tubing confinement. The concept relies upon the two different material states of the differing material properties of the superelastic metal in the martensitic state and the austenitic state, as well as the properties within the transformational temperature range (i.e. from A s (Austenitic start) temperature to A f (Austenitic finish) temperature) for the expandable material composition of the device. In the Martensitic state, nitinol is relatively pliable and it is not superelastic. While in the Martensitic state at lower temperatures, it has a relatively low modulus of elasticity (compliant), while in the fully austenitic state it is superelastic and it has a relatively high modulus of elasticity (stiff).

It is proposed the relatively thin walled and/or slotted constraining tubes that house the superelastic self-expanding shape set device can be additionally constrained by an encircling larger diameter tubing (plus or minus circumferential in configuration, but in a preferred implementation, circumferential), during inventory storage and transport of the device. Immediately prior or just prior to surgical application (e.g. within the preceding day, hours, or minutes) the device and in particular the components of the shape set superelastic material can be brought to a reduced temperature (relative to ambient) to condition the material in the transformation temperature range (e.g. refrigerated or immersed in a chilled or ice bath). With the material in the transformation zone temperature range, the most outer constraining tubular element can be removed, providing for adequate constraint from the thin walled and/or slotted tubing due to the reduced outwardly expanding force exerted by the chilled shape set and only partially superelastic material. For commonly used nitinol material this can be in the temperature range from −4 degrees Fahrenheit to 50 degrees Fahrenheit.

In addition it is contemplated the constraining elements either or both might have thermally insulating material disposed about their surfaces or as a coating to retard the warming effects of exposure of the chilled implant/delivery device once the most exterior constraining element has been removed. Once delivered, the implant can be warmed by local body heat or heated by various means (e.g. irrigation with warmed physiologic solutions) to facilitate transformation into the shape set superelastic state. The most external constraining element or tube can be associated with the device only during storage and can be removed or pulled off the distal tip of the device after bringing the devices shape set material to a lower than ambient temperature with various chilling means (e.g. refrigeration or chilling bath) while in the immediate operative setting or immediately prior to surgery. This can allow for the use of a thinner walled and/or slotted constraining tube immediately surrounding the nitinol during surgical delivery. An implementation of the previously described variant includes the use of a physiologic solution to warm the deployed device to promote expansion in situ.

The plurality of struts of the devices described herein can provide the body with a defining perimeter having various shapes. The shape of the expanded anchor device can vary depending on the region in which the anchor device is expanded. Generally, the plurality of struts expands outward from the longitudinal axis A of the device such that they take on a curved or otherwise bowed shape. The plurality of struts can bow radially outward from a central axis A of the anchor device such that the perimeter of the expanded anchor is generally conical in shape. Each of the struts can expand to a greater extent near the proximal end region of the device compared to the distal end region of the device (see, for example, FIG. 7A). The amount of expansion and extension of the struts away from the longitudinal axis A near the proximal end region of the anchor device aid to resist pull-out of the anchor device from the bone. The plurality of struts also can expand outward such that a portion of the struts is bent to a certain angle giving the device a more angular perimeter shape. The plurality of struts can provide the device with a variety of perimeter shapes including fusiform, oblong, spheroid, umbrella, oval, wedge, cone, frustoconical, pyramidal, triangular, half-moon, or other shape that can be symmetrical or asymmetrical. It should be appreciated that the plurality of struts in the devices described herein can be disposed symmetrically or asymmetrically around a central axis of the device.

As described herein, the surface geometry of the anchor devices can be generally discontinuous such that a plurality of slots defines the plurality of struts. The width of the plurality of slots can vary resulting in variable widths of each of the plurality of struts. Further, the number of the slots and thus, the number struts can vary. The struts can be made thicker or thinner to achieve a particular strength for a particular purpose. Further, the thickness of each of the struts can vary along their length such that a portion near a distal end region or a proximal end region is thicker than a centrally disposed portion of the strut. Each of the struts can have a wider, more flattened configuration or can have a more rounded wire-like configuration. The wall thickness and width of the struts can be uniform or non-uniform.

As described above the anchor devices described herein can be configured to anchor soft tissues such as tendon or other tissue to or within a rigid material such as cortical bone. Some anchor devices include one or more attachment features that facilitate securing or coupling of materials to the anchor device (see, e.g. attachment feature 40 shown in FIG. 3, attachment feature 430 shown in FIG. 4, or attachment feature 500 shown in FIG. 5). It should be appreciated that the anchor devices need not incorporate an attachment feature and can anchor soft tissues relying upon interference fit between one or more regions of the device, the soft tissue and the defect.

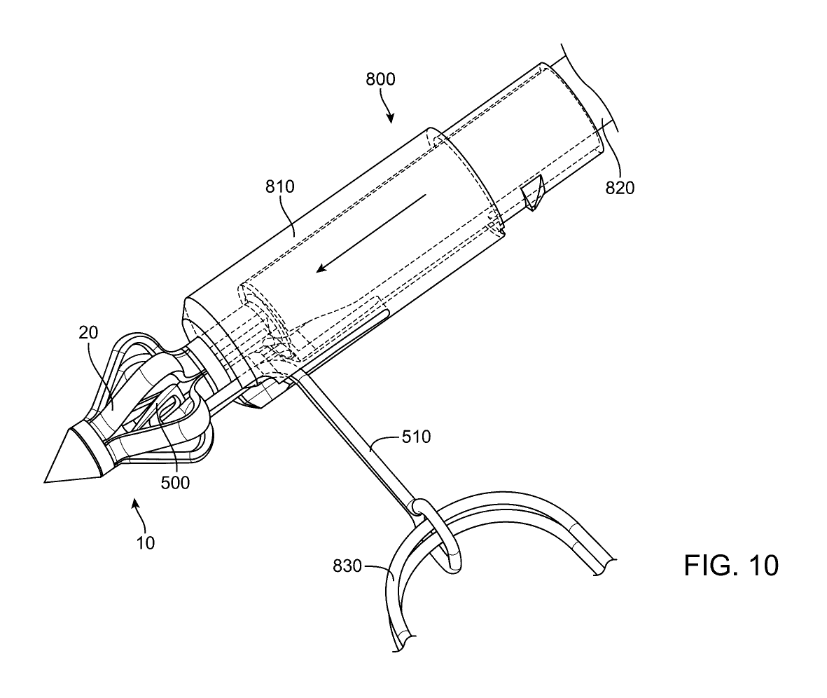

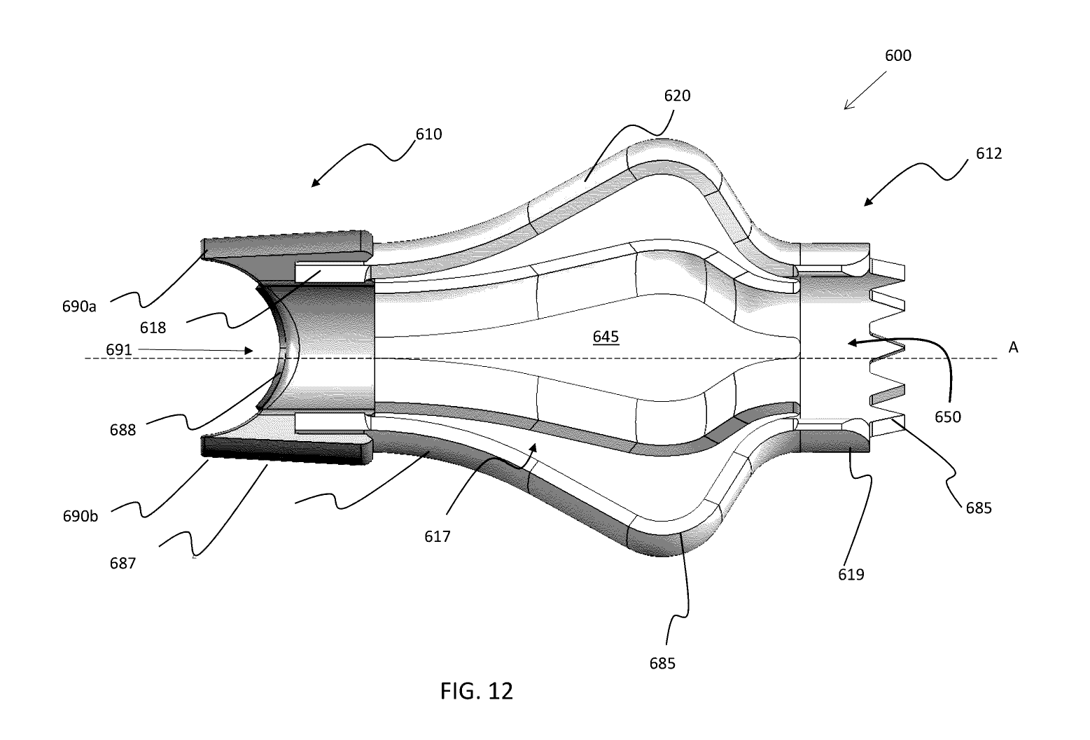

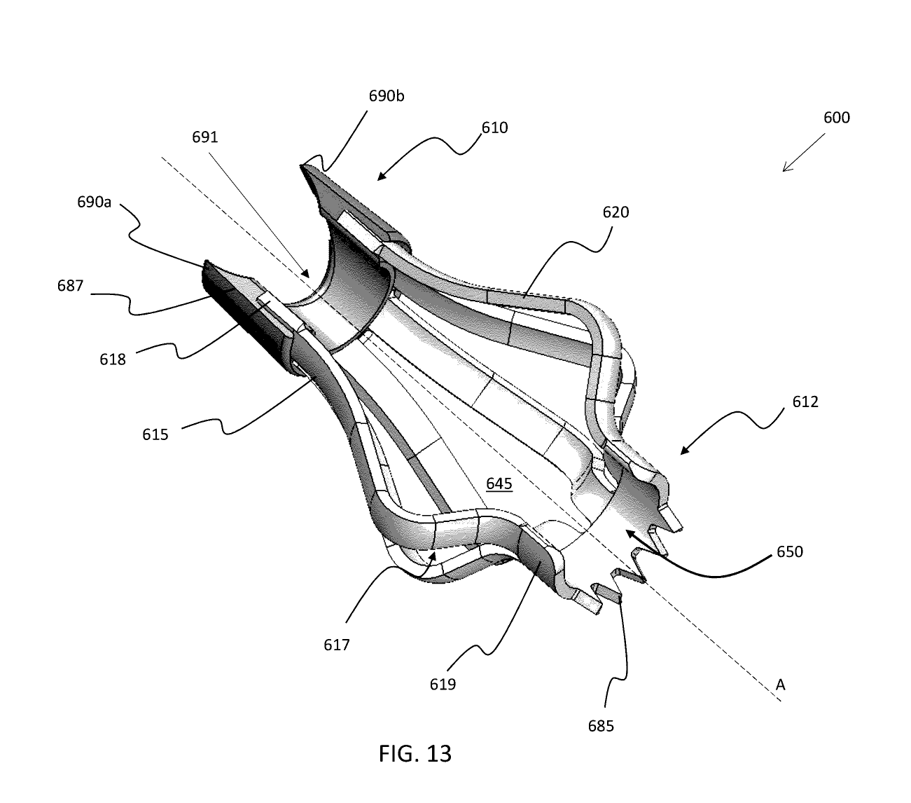

FIGS. 11-18 show an interrelated implementation of an anchor device 600. As with other implementations described herein, the anchor device 600 can include a body 615 having a plurality of slots 617 forming a plurality of stays or struts 620 at least partially surrounding an internal volume 645 of the body 615. The anchor device 600 can include two or more longitudinally extending slots 617 along its long axis A from a distal end region 610 to a proximal end region 612 on its body 615 that can define the edges or margins of the deployable struts 620. The anchor device 600 can employ similar radially expanding features to secure the anchor device 600 with the medullary cavity of the bone as described above.

The anchor device 600 can be used to deliver and secure a generally cylindrical segment of a detached tendon's terminus within a closely confining bore or channel of bone 601. As with other implementations of anchor devices described herein, the anchor device 600 can provide for limited contact with the surface features of the reattached soft tissue to optimize the biological repair process. As will be described in more detail below, cradling the soft tissue 605 to be affixed within the highly porous architecture of the device 600 approximates the soft tissue 605 to the bone 601 in a manner that limits the compression of the soft tissue 605 against the cortical rim and affords abundant opportunity for vascularization of the soft tissue 605 as well as fibrous and boney attachments to the perimeter of the soft tissue 605.

The body 615 can in its constrained configuration be generally cylindrical having a plurality of slots 617 extending through the wall of the body 615 forming the plurality of struts 620. The plurality of slots 617 can be generally shorter in length than the overall length of the body 615 such that an outer wall 618 can be formed at the distal end region 610 of the body 615 and an outer wall 619 can be formed at the proximal end region 612 of the body 615.

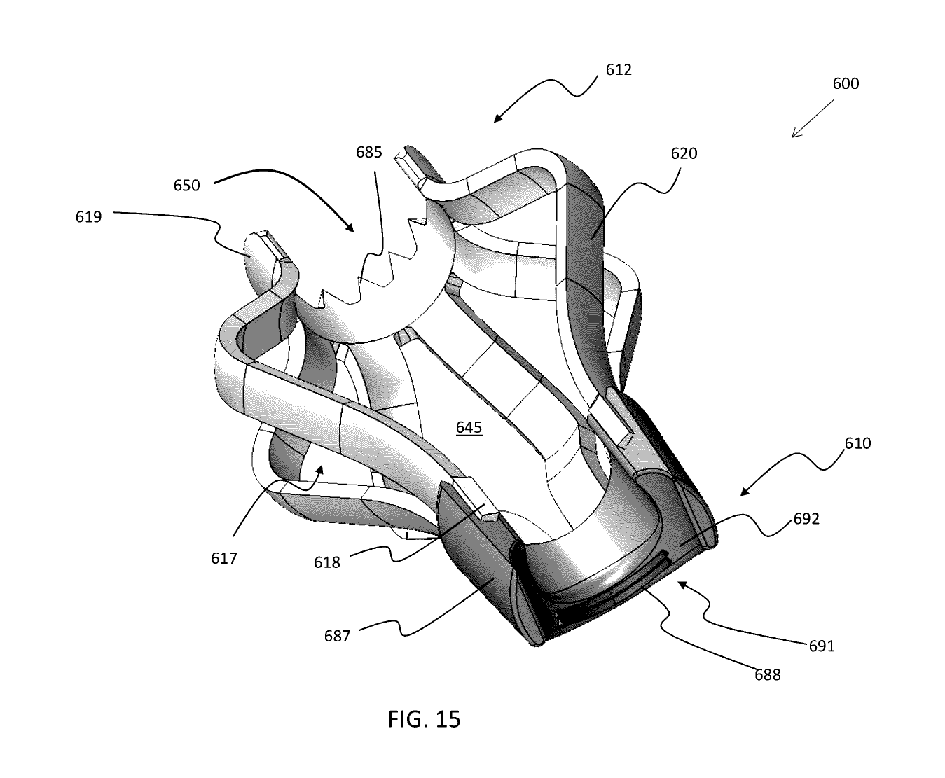

The outer wall 619 at the proximal end region 612 of the body 615 can form or define the proximal opening 650 into the internal volume 645 of the body 615. The outer wall 619 at the proximal end region 612 of the body 615 can be discontinuous forming a gap such that in cross-section it defines a generally c-shaped proximal opening 650 to the internal volume 645. Upon deployment of the anchor device 600, the discontinuous proximal outer wall 619 can be positioned near or within the bone channel through which the anchor device 600 was delivered (see FIGS. 16-18). The gap or circumferential discontinuity in the proximal outer wall 619 of the otherwise cylindrical shape of the body 615 allows for at least a portion of the soft tissue terminus 605 being delivered to be placed in direct intimate contact with the channel or cortical defect in the bone 601 and medullary cavity of the bone, particularly the closely sized cortical defect, promoting tendon to bone healing. The gap in the proximal outer wall 619 can also provide for easier introduction of the soft tissue terminus 605 within the cavity or channel of the device and allow for asymmetric deployment/expansion of the device. Having the soft tissue terminus 605 exposed along the circumference of the tendon-device construct and sizing the cortical defect (e.g. drill hole) to provide for an intimate fit, can enhance not only the initial fixation but can also improve long term tendon biological fixation.

The proximal end region 612 of the device 600 can form an edge having a plurality of serrations 685. When in use, the serrations 685 can be adjacent to the region in which the soft tissue 605 is compressed between the implant 600 and the cortical bone 601 (see FIG. 16). These sub-cortical serrations 685 can impale and/or catch on the soft tissue 605 just proximal to the region of the soft tissue 605 secured by trapping the soft tissue 605 between the device 600 and the cortical bone 601 helping to prevent or reduce soft tissue migration.

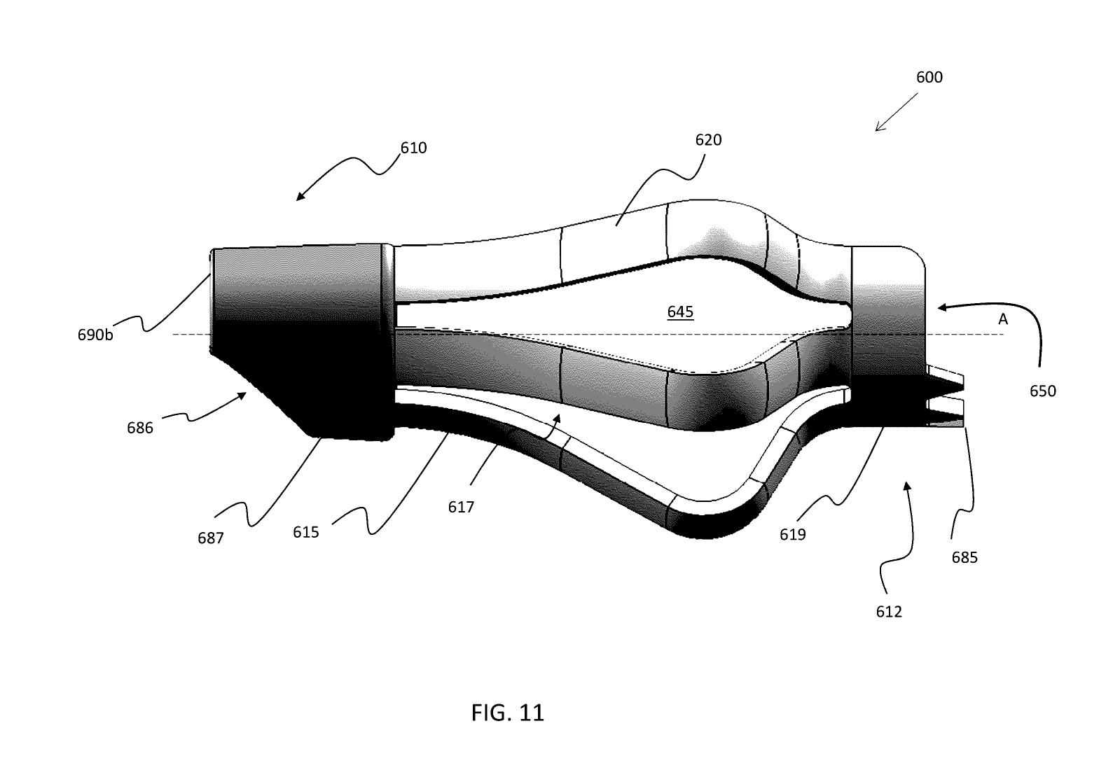

The distal end region 610 of device 600 can be open such that the internal volume 645 extends the entire length of the anchor device 600 between the proximal end region 612 and the distal end region 610. The soft tissue 605 can extend clear through the entire length of the internal volume 645 and loop around the distal end region 610. The distal end region 610 of the device 600 can include a cap 687 covering the outer wall 618. The cap 687, like the distal outer wall 618 of the body 615, can be c-shaped in cross-section such that one side of the cap 687 is open and the opposite side of the cap 687 forms a surface against which the soft tissue 605 can fold over when it loops around the distal end region 610. As best shown in FIG. 12, the distal outer wall 618 of the body 615 can insert within the proximal-most end of the cap 687 such that the cap 687 covers the inner and outer surfaces of the distal outer wall 618 of the body 615. The proximal end of the cap 687 and the distal outer wall 618 of the body 615 have inner and outer surfaces that are generally cylindrical and form the c-shape.

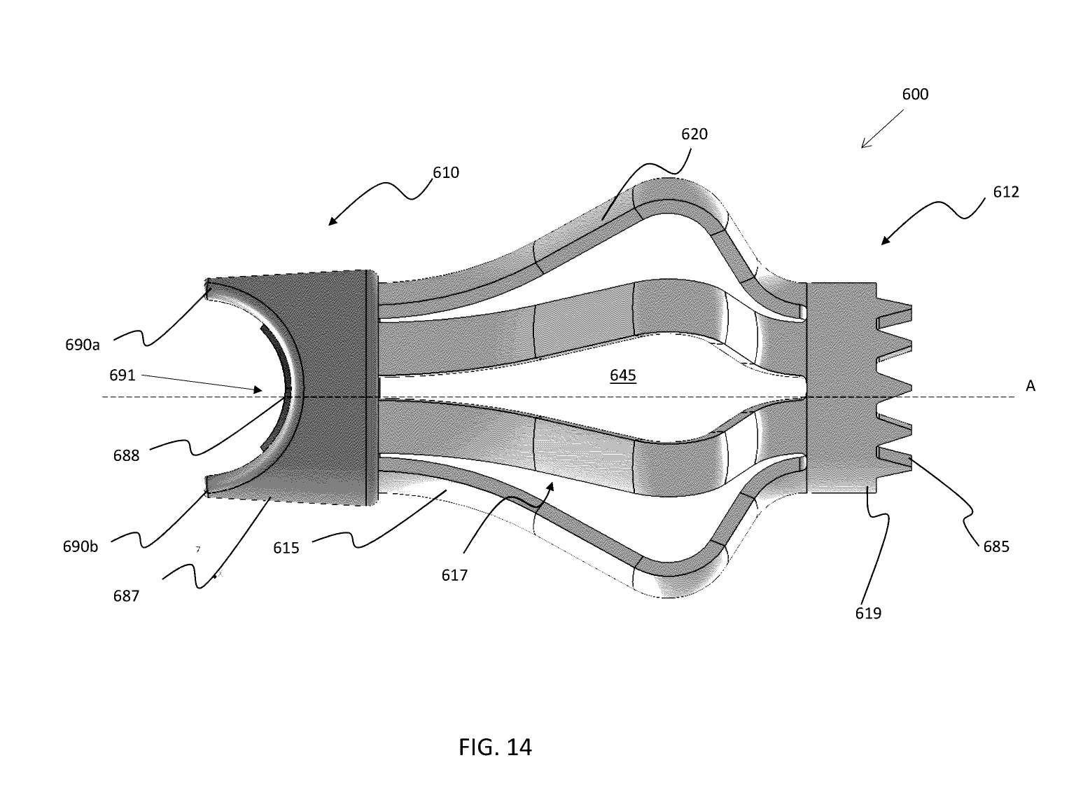

The cap 687 can extend distal to the distal outer wall 618 of the body 615 forming a notch 686 (see FIGS. 11-12). The notch 686 in the cap 678 can support and redirect the soft tissue within the bone such that the cap 678 can constrain the redirected soft tissue 605 and distribute loading of the soft tissue 605 on the notch 686 in a manner that is gentler to the soft tissue 605 than the bare edge of the device 600. The notch 686 can have two opposed, distal-extending prongs 690a, 690b having a saddle region 691 therebetween (see FIGS. 12, 13, and 14). The cap 687 can have a surface edge 692 near its distal-most end within the saddle region 691 that has a dimension broader than the bare edge of the distal outer wall 618 (see FIG. 15). One or more ridged surfaces 688 can be located within the notch 686, such as on the surface edge 692 of the saddle region 691. The one or more ridged surfaces 688 can increase surface friction and drag preventing tendon migration. The ridged surfaces 688 can be oriented in a direction that is transverse to the longitudinal axis A of the device 600. In some implementations, the ridged surfaces 688 can be molded into the material of the cap 687. The cap 687 can be formed of a material or materials that are softer than the material of the distal end region 610 of the device 600. In some implementations, the material of the cap 687 can include a polymeric material such as unfilled polyetheretherketone (PEEK).

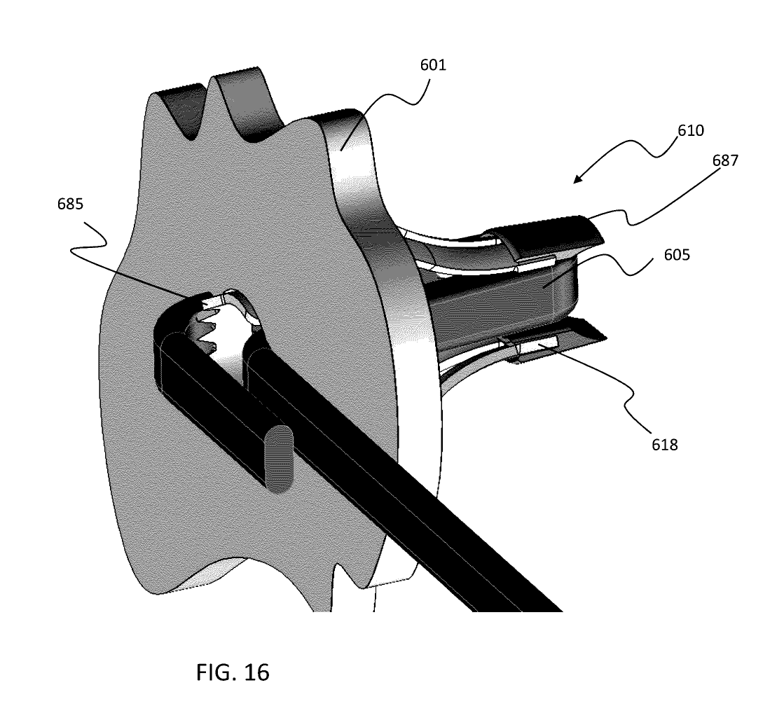

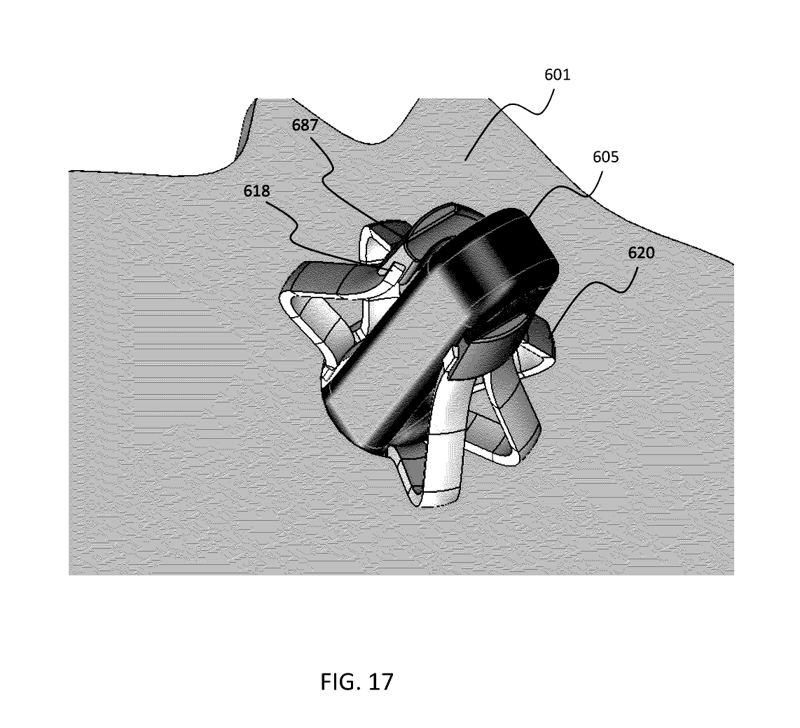

FIG. 16 illustrates a perspective view of the anchor device of FIG. 11 anchored into bone. A “free” or detached end of soft tissue is shown trapped between the wall of the bone cortex 601 and the device 600 (see also FIG. 18). A “muscle side” of the soft tissue 605 is shown exiting from an intraosseous location from within the interior volume 645 of the device 600. The soft tissue 605 is shown wrapped around a distal end region 610 of the imbedded device 600 (best shown in FIG. 17). The cap 687 is redirecting the soft tissue 605 along a deep surface of the cortical bone 601. The stays 620 of the device 600 are expanded retaining the device 600 within the subcortical bone 601.

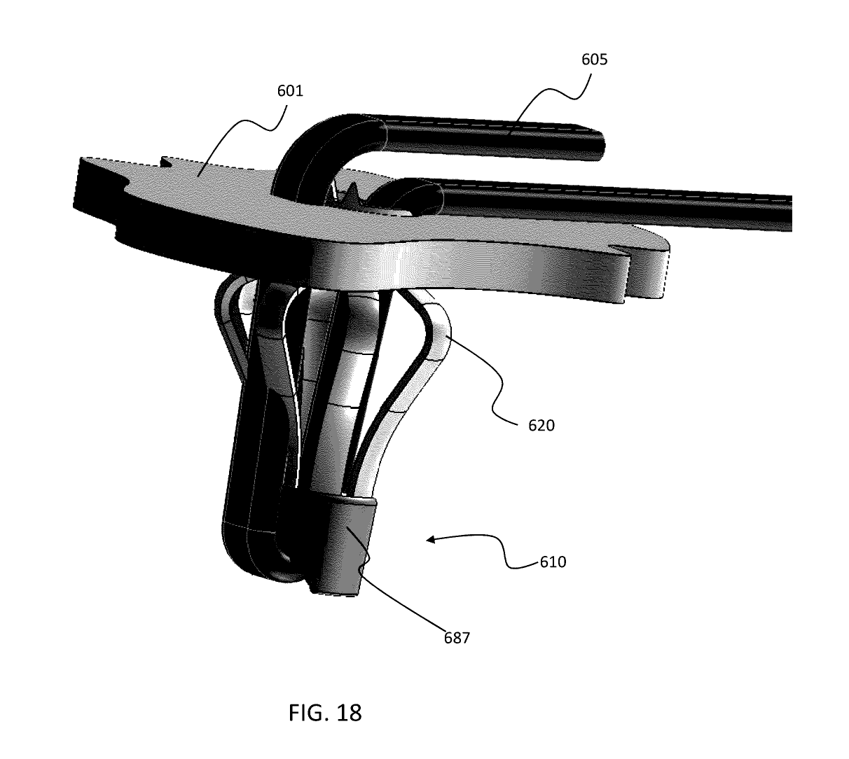

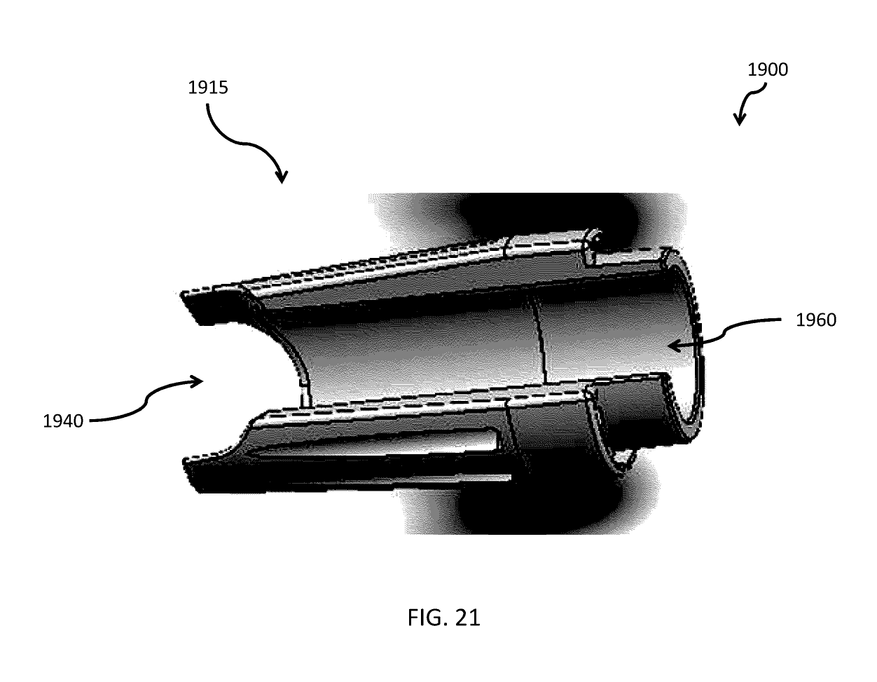

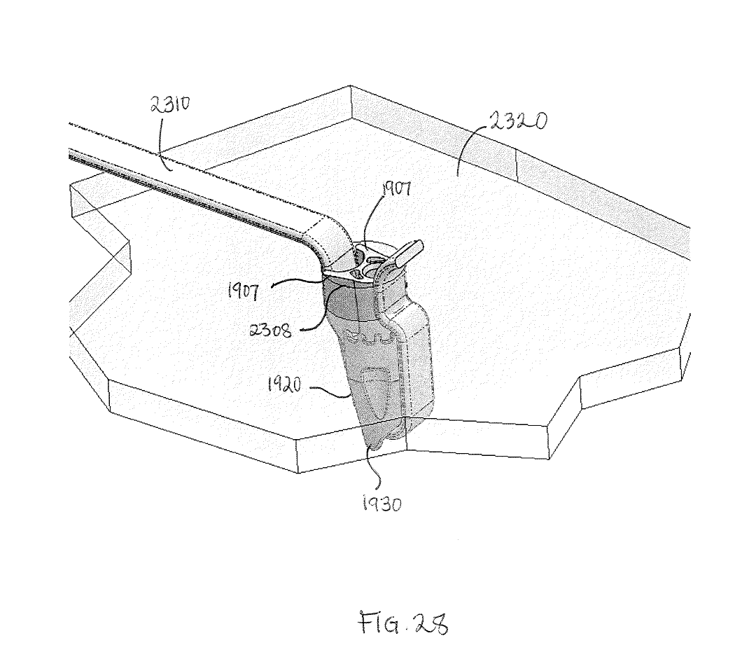

FIGS. 19-30 illustrate an interrelated implementation of an anchor device. The anchor device 1900 has a body 1915 having a distal end region and a proximal end region each at least partially surrounding an interior volume forming a slot extending generally along a longitudinal axis of the body. The anchor device 1900 has a concave saddle surface at the distal end region of the body configured to receive and redirect a soft tissue segment extending through the slot in a first direction from the proximal end region of the body to a second direction that is at an angle to the first direction. As will be described in more detail below, the body 1915 is configured to be transiently constricted from a relaxed state having a first outer dimension to a constricted state having a second outer dimension that is smaller than the first outer dimension by a cortical aperture within the bone such that a soft tissue segment to be anchored is trapped between at least a portion of the body 1915 and at least a portion of the bone cortex upon relaxation of the body towards the relaxed state subcortically. The anchor device 1900 can fix soft tissue to bone by placing the anchor device superficial to a segment of soft tissue, such as a tendon or a ligament, superficially juxtaposed to a unicortical blind hole in a bone, the anchor device having a relaxed state characterized by a first outer dimension. The soft tissue segment is driven into the blind hole using the anchor device. The anchor device transiently constricts to a constricted state during insertion through the blind hole, the constricted state characterized by a second outer dimension that is smaller than the first outer dimension. The portion of the soft tissue segment is trapped between a portion of the anchor device and a portion of the bone when at least a portion of the anchor device is disposed sub-cortically and relaxes towards the relaxed state. The distal end region of the body 1915 can be positioned sub-cortically to the bone defect and at least a portion of the proximal end region of the body can be positioned intra-cortically within the bone defect.

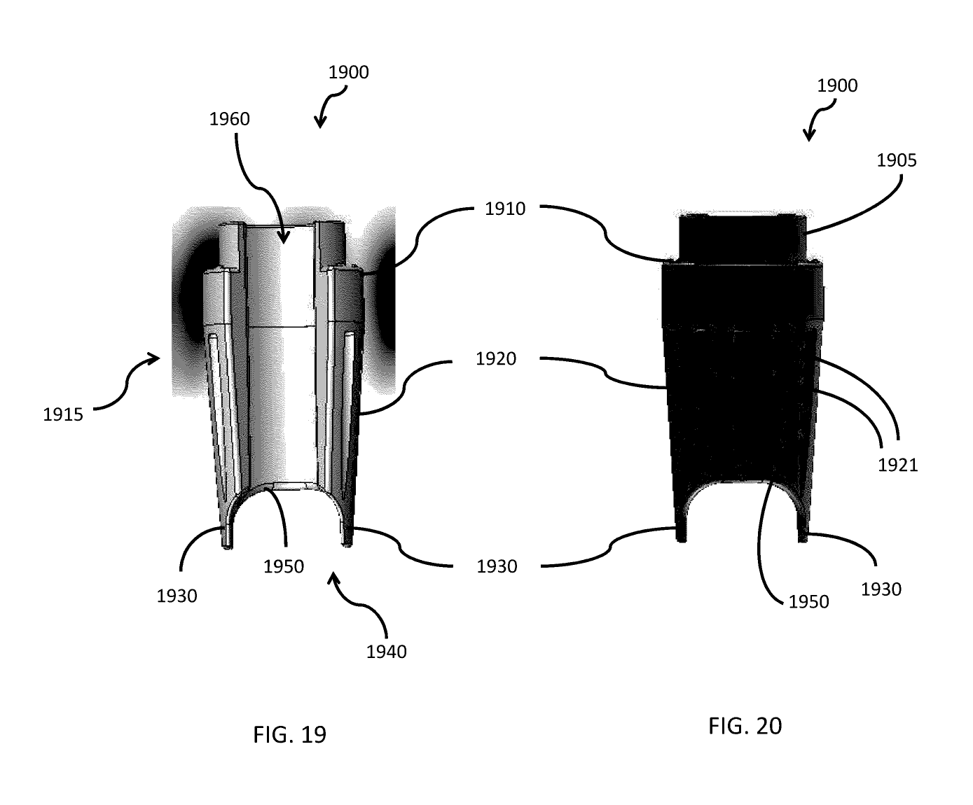

The anchor device 1900 can include a proximally-extending cortical feature 1905. The cortical feature 1905 is configured to be positioned at least in part within a cortical defect 2308 of bone 2320 and the body 1915 is configured to be positioned subcortically. The body 1915 together with the cortical feature 1905 defines a slot 1960 extending from a proximal end region of the device 1900 to a distal end region of the device 1900. The presence of the slot 1960 provides a generally C-shape or arcuate cross-sectional geometry to at least a portion of the body 1915, and/or both the cortical feature 1905 and the body 1915 of the device 1900. The c-shaped cross-section can be dimensioned to control the outer dimension achieved in the constricted state of the device during insertion into a bone defect. The arcuate length and cross-sectional size of the cortical feature 1905 can be smaller than the arcuate length and cross-sectional size of the proximal end region of the body 1915 such that a cortical rim retention shelf 1910 is formed near the proximal end region of the device 1900. The relationship between the cortical rim retention shelf 1910 and the cortical feature 1905 can create an offset that serves to trap and redirect the soft tissue structure under the cortical margin of the bone bore 2308. The body 1915 can taper from the cortical rim retention shelf 1910 to the distal end region, forming a substantially hollow, generally conical or frustoconical shaped device having a tapered side wall 1920 (see FIGS. 19-20). The body 1915 can have a generally hemi-frusto-conical geometry and an arcuate length at the proximal end region of the body 1915 that is greater than an arcuate length at the distal end region of the body 1915.

As with other implementations of anchor devices described herein, the anchor device 1900 can provide for limited contact with the surface features of the reattached soft tissue to optimize the biological repair process. The slot 1960 can allow a soft tissue structure 2310 (such as a tendon or other soft tissue to be attached to bone) to pass through and be accommodated within the body 1915, for example, while suspending the soft tissue structure 2310 within the intramedullary canal for subsequent healing. Again with respect to FIGS. 19-20, at the tapered distal end, the anchor device 1900 can include a pair of opposed, distally-projecting confinement prongs 1930 on either side of the midline axis. The confinement prongs 1930 and the body 1915 can define a recessed region 1940 forming a concave saddle-type geometry or a soft tissue redirecting distal saddle surface 1950 at the distal end of the anchor device 1900. The concave saddle surface 1950 can be configured to receive and redirect the soft tissue segment extending through the slot 1960. The saddle surface 1950, for example, can redirect the soft tissue segment extending through the device in a first direction from the proximal end region of the body to a second direction that is at an angle to the first direction as will be described in more detail below. In some implementations, the distal saddle surface 1950 can be provided with (or without) friction enhancing ridging or similar friction enhancing ridged surfaces as described elsewhere herein. As with other implementations described herein, the anchor device 1900 can be used to deliver and secure a looped segment of soft tissue 2310 such as a tendon terminus or other tissue to or within a rigid material such as cortical bone 2320 having a closely confining bore 2308 or channel of bone. The confinement prongs 1930 are configured to straddle soft tissue 2310 on insertion and with fixation forming laterally-confining elements within which the soft tissue 2310 courses around the distal end of the body 1915 during redirection to be discussed in more detail below.

The anchor devices described herein need not incorporate a plurality of radially expanding stays or struts to secure the anchor device with the medullary cavity of the bone as described above and as shown in the implementations of FIGS. 1-18. Rather, the anchor devices described herein can be temporarily constricted to a more narrow configuration during insertion through the bone defect and subsequently relax back into a geometry of larger cross-section following insertion to provide interference fit within the cortical defect. In lieu of expanding struts (for example, as shown in the other implementations described herein), the cortical rim retention shelf 1910. During advancement of the device 1900 within a cortical channel or bore 2308 that has an overall smaller radial dimension compared to the device 1900, the generally cross-sectional C-shaped body can allow for hinged and radial contraction. The flexure or hinging of the device 1900 can occur along the longitudinal axis of the device as the frustoconically-shaped device is forced within and constricted by a cortical hole of smaller radial dimension and constrained by the loop of soft tissue. The implant can hinge around the longitudinal axis of the device (i.e. closing or narrowing the c-shape). In addition, as the device is displaced and constrained by the cortical defect and the soft tissue loop, the device can experience some radial contraction. The anchor device 1900 can be configured to flex during insertion (e.g., driven into a circular cortical defect 2308 using an insertion tool 2300) in a radial and arcuate manner, resulting in the slightly oversized anchor device 1900 (oversized with respect to the cortical defect 2308 having a slightly smaller diameter and competing with the tendon terminus loop within that defect) temporarily collapsing and/or undergoing “arcuate constriction.” The anchor device 1900 can also be configured to elastically recoil or re-expand within the medullary cavity under the cortical rim. For example, in some implementations, the cortical rim retention shelf 1910 near the proximal end region of the body 1915 can be sized with a slightly larger diameter than the diameter of the cortical defect 2308 so as to provide tendon fixation and secure subcortical retention of the anchor device 1900 to or within the cortical bone 2320.

The flexibility/resiliency of the anchor device 1900 can be provided in various ways. For example, the monolithic structure of the anchor device 1900 can be provided with a degree of flexibility/resiliency through a flexible/resilient material and/or a non-uniform wall thickness of the circumferential configuration of the C-shaped body 1915 allowing its maximum diameter to be reduced during insertion into a comparatively smaller defect. The body 1915 can be formed of an elastomeric material or combination of materials, such as polyetheretherketone (PEEK) polymer, ultrahigh molecular weight polypropylene (UHMWPE), Nitinol alloy and others. The transverse arcuate wall thickness of the body 1915 can be non-uniform. The transverse arcuate wall thickness can be greater near one or both of the margins compared to a central region. The body 1915 can include one or more scalloped features 1921. The scalloped features 1921 can have a variety of shapes and sizes, but are generally relatively shallow features disposed longitudinally along the body 1915. The scalloped features 1921 can be located equidistant from the lateral edges or sides of the implant. The scalloped features 1921 can reduce the wall thickness in portions of the body 1915. The interruption provided by the scalloped features 1921 can be dimensioned to accommodate (and/or to control) the desired reduction in diameter during flexure to facilitate the insertion of the anchor device into the cortical defect. The scalloped features 1921 can also prevent molded polymer part “sink” or retract from its molded dimensions.

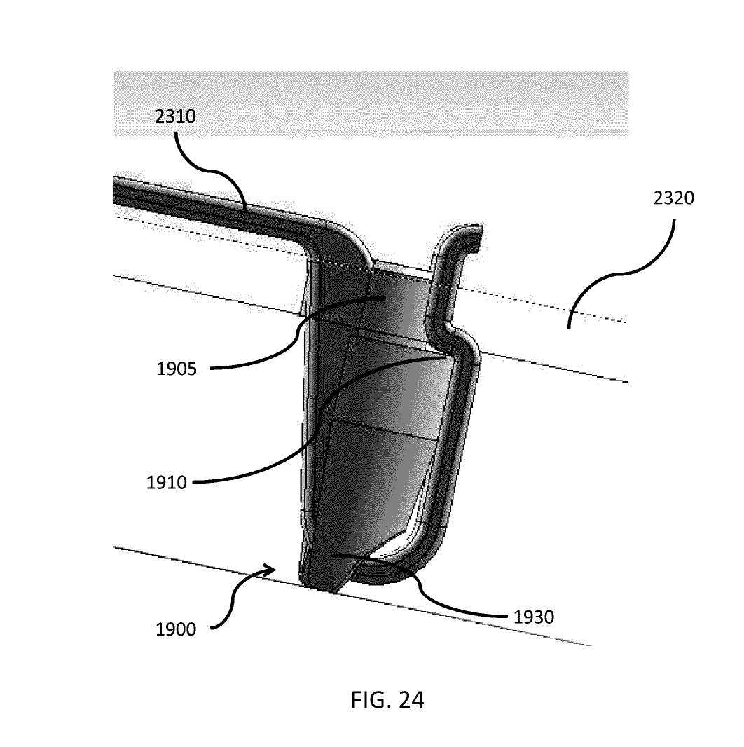

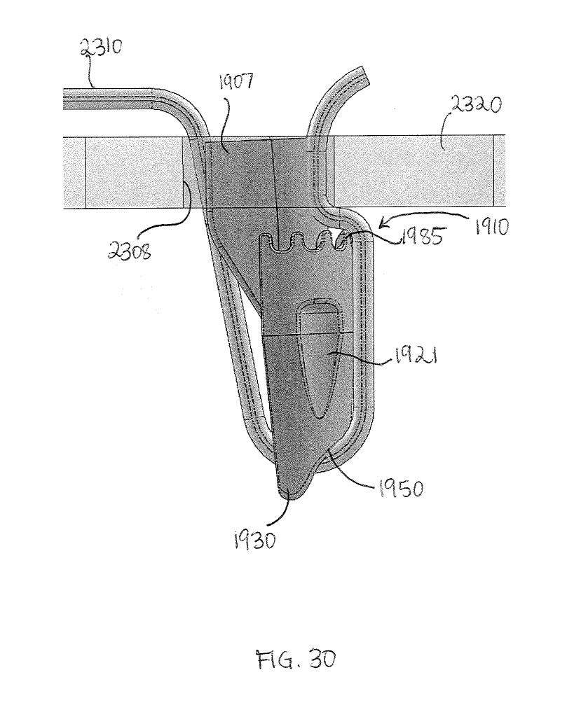

As mentioned above, the cortical rim retention shelf 1910 near the proximal end region of the device 1900 can be configured for deployment deep to the cortical rim of a cortical defect 2308 through which a soft tissue structure 2310 is to be fixated. In some implementations, the cortical retention shelf 1910 can be configured with an external geometry that is a segment of a circumference corresponding to a radius that is larger than the radius of the cortical defect 2308 through which the device 1900 and a soft tissue structure 2310 are to be inserted. As such the arcuate length of the proximal end of the body 1915 can be greater than a diameter of the bored defect 2308. As the device 1900 is inserted into the defect 2308 and the C-shape undergoes flexure and hinging, the proximal aspect of the shelf 1910 can return towards its resting state upon passing distally beyond the rim of the cortical defect 2308 leaving at least a portion of the cortical feature 1905 within the defect 2308 (see FIGS. 24 and 30).

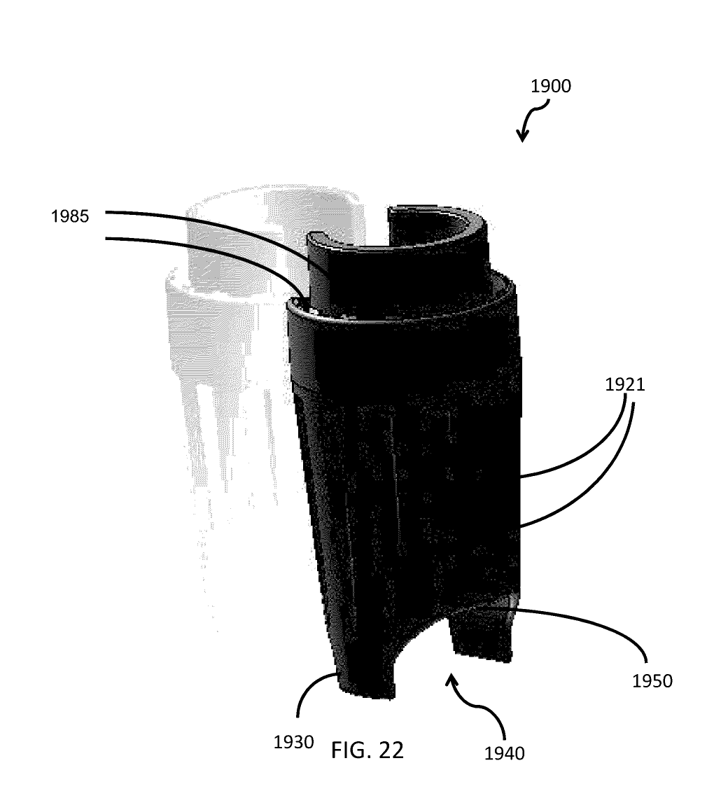

The proximal aspect of the cortical rim retention shelf 1910 can form an edge having a plurality of spikes, serrations, or one or more projecting elements 1985 (see for example FIG. 22 and FIGS. 25-30). The projecting elements 1985 can form a continuous or discontinuous arcuate pattern on the proximal or superficial surface of the retention shelf 1910 to provide additional tendon fixation and enhance the fixation provided by interference fit. When in use, the projecting elements 1985 can engage the redirected soft tissue structure 2310 trapping it against an interior surface of the cortical bone 2320 within which the device 1900 is deployed. These sub-cortical projecting elements 1985 can impale and/or catch on the soft tissue 2310 just proximal to the region of the soft tissue secured by trapping the soft tissue between the device 1900 and the cortical bone 2320 helping to prevent or reduce soft tissue migration.

As mentioned above, the device 1900 can also include a proximal cortical feature 1905 extending proximal to the cortical rim retention shelf 1910. Where the cortical rim retention shelf 1910 is configured to be inserted within the sub-cortical region, at least a portion of the cortical feature 1905 remains positioned within the defect 2308. The proximal cortical feature 1905 can have an external surface 1903 configured to trap the divided or detached end of the tendon or soft tissue structure 2310 against a surface of the defect 2308. In some implementations, the external surface 1903 of the cortical feature 1905 can be configured with a geometry that corresponds with a radius that is equal to or slightly smaller than the cortical defect 2308 through which the device 1900 and the looped soft tissue segment 2310 are inserted. At least a portion of the external surface 1903 of the cortical feature 1905 can provide compression of the soft tissue between the anchor device 1900 and the internal cortical rim of the cortical bone defect 2308 once the device 1900 is implanted.

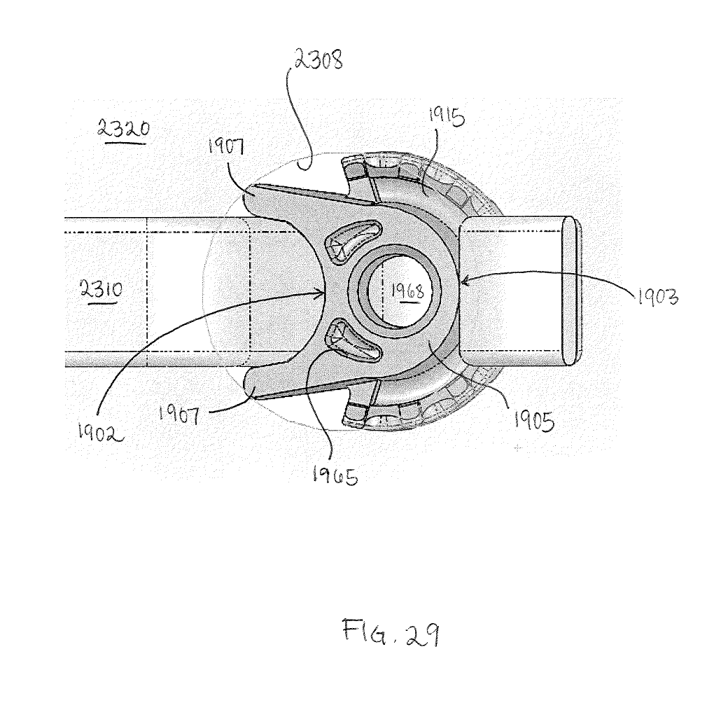

In some implementations, the cortical feature 1905 can also include a pair of cortical interference fit extensions 1907. The extensions 1907 are located on a region of the cortical feature 1905 extending away from the external surface 1903 configured to compress or trap the detached end of the tendon (see FIG. 29). The extensions 1907 can surrounding a concave surface 1902 configured to at least partially contain the attached end of the soft tissue structure (e.g. the muscle end of a tendon) and direct the soft tissue structure into the slot 1960. As shown in FIG. 30, a proximal end of the extensions 1907 can reside, at least in part, within the cortical rim aperture 2308 and a distal end of the extensions 1907 taper inward down towards the sub-cortical region and the tapered walls 1920 of the body 1915. The soft tissue segment 2310 can course from a superficial side of the cortical defect 2308 between the two extensions 1907 before the soft tissue segment 2310 passes deep within the bone bore 2308 along the concave aspect of the body 1915. Thus, the interference fit extensions 1907 can help to create a first redirection of the soft tissue segment 2310, which will be described in more detail below.

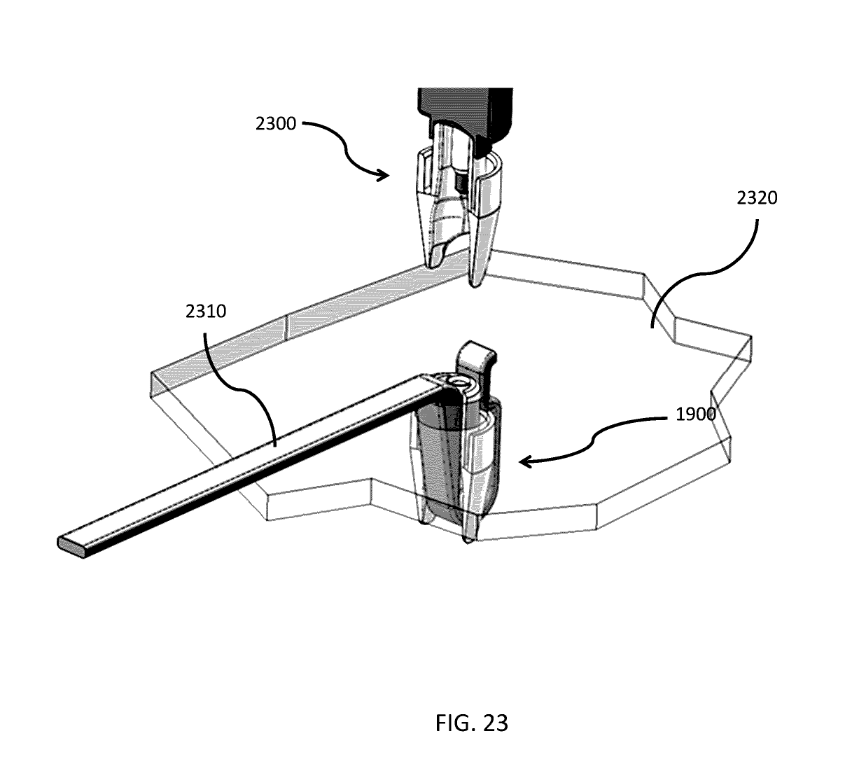

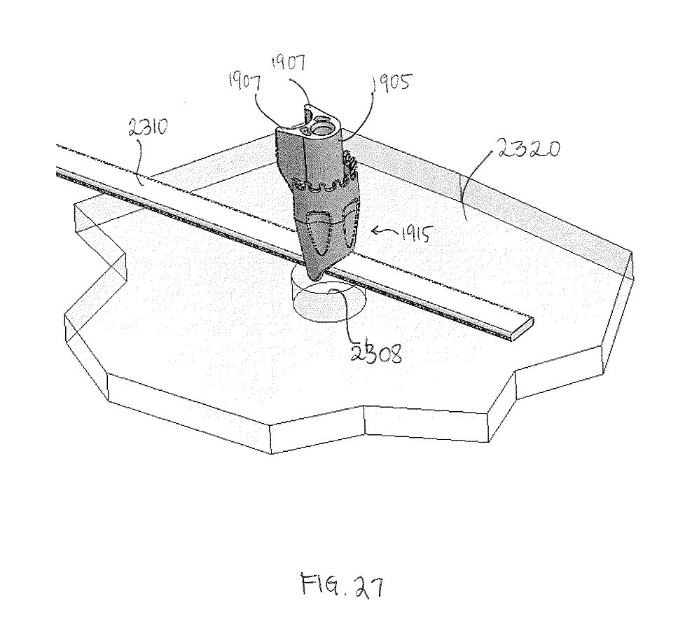

As shown in FIGS. 23 and 24 and also FIGS. 27-30, the device 1900 can be driven into a cortical cancellous bore 2308, which as described elsewhere herein, results initially in a radial contraction and arcuate hinging of the device through a region of the cortical bone with subsequent relaxation of the device 1900 in a region of subcortical bone. The anchor device 1900 can include a body 1915 that is relatively pliable and elastic compared to the bore 2308 through which it is inserted. The body 1915 can have a generally arcuate-shaped transverse cross-sectional geometry, a generally orthogonally-oriented long axis, distal and proximal ends. As mentioned above, the transverse arcuate length of the device can be shorter near the distal end compared to the transverse arcuate length near the proximal end providing an overall tapered geometry to the body 1915. The distal end of the device 1900 can be positioned within a depth of the bone's medullary cavity and the proximal end region positioned near the cortical surface of a bored defect 2308 within the bone 2320. The soft tissue structure 2310 can be positioned within the bored defect 2308 of the bone 2320 such that the soft tissue structure 2310 undergoes two or more redirections in combination with compression and interference fixation within the cortical defect 2308. For example, the soft tissue structure 2310 can course along the long axis of the device 1900 on a concave surface of the device (e.g. the surface of the slot 1960), sharply redirected by wrapping around a distal edge of the device 1900, and coursing along the long axis of the device 1900 on a convex surface of the device 1900 (e.g. outer surface of the body 1915). The distal saddle surface 1950 (with or without ribbing to increase friction) of the distal aspect of the device 1900 can serve to redirect the soft tissue structure 2310 within the bone bore 2308 with fixation. After the course of the soft tissue structure 2310 is redirected around the distal edge back in a superficial direction, it can be redirected again at the proximal end region in the subcortical aspect of the device 1900. The anchor device 1900 redirects (e.g., angles) the soft tissue 2310 at the subcortical aspect around an edge of the cortical rim retention shelf 1910 and redirects (e.g., angles) the soft tissue 2310 again at the juncture of the retention shelf 1910 with the cortical feature 1905, along the external surface 1903 of the cortical feature 1905. The segment of the detached end of the soft tissue structure 2310 can be compressed and pinned between the cortical surface (i.e. cortical rim) of the bored defect 2308 and the external surface 1903 of the cortical feature 1905 (see FIG. 24 and FIG. 30). It should be appreciated that the interactive contact between the soft tissue structure 2310 and the various surface features of the device 1900 redirects the soft tissue structure fibers at least twice (i.e. into and out of the unicortical blind hole in the bone). However, the soft tissue structure can be redirected more than twice, such as three, four or more redirections within the defect. The first redirection of the soft tissue structure into the cortical defect 2308 can be from an orientation that is generally parallel with the cortical plane from a superficial side of the cortical defect 2308 to an axis that is relatively perpendicular to the cortical plane (within the bone bore 2308). The first redirection can be aided by the interference fit extensions 1907. The second redirection of the soft tissue structure within the cortical defect can be nearly 180 degrees around the distal saddle surface 1940 from a sub-cortical level back towards the cortical defect. The third redirection of the soft tissue structure can be nearly 90 degrees in the subcortical region by a proximal aspect of the hollow truncated cone of the body 1915 back through the cortical defect and can be aided by the proximal plurality of serrations 1985. The fourth redirection can be nearly 90 degree such that the soft tissue segment 2310 is trapped, resides, and/or is compressed between the semi-circular, convex external surface 1903 of the cortical feature 1905 and the cortical rim of the bone defect 2308. Redirecting the looped soft tissue segment around several edges/surfaces of the implant as well as the cortical rim of the bone enhances the fixation provided by interference compression between the implant and the cortical rim. The redirections can increase the friction between the device 1900 and the soft tissue structure 2310 to avoid tensile loads directly on the segment of the soft tissue structure that is being trapped between the cortex and the proximal aspect of the device. The redirections also expose a long segment of the soft tissue surfaces to the cortical and subcortical aspect of the bone bore 2308 for biologic integration. It should be additionally appreciated that a majority of the soft tissue 2310 that is being trapped within the bone bore 2308 is not being compressed from the cortical entry position to the subcortical position. Avoiding compression along this length of the soft tissue 2310 helps to avoid pressure necrosis and inadvertent transection that can occur. It should also be appreciated that the actual angles achieved by the various redirections can vary and the angles provided herein are simply for illustrative purposes.

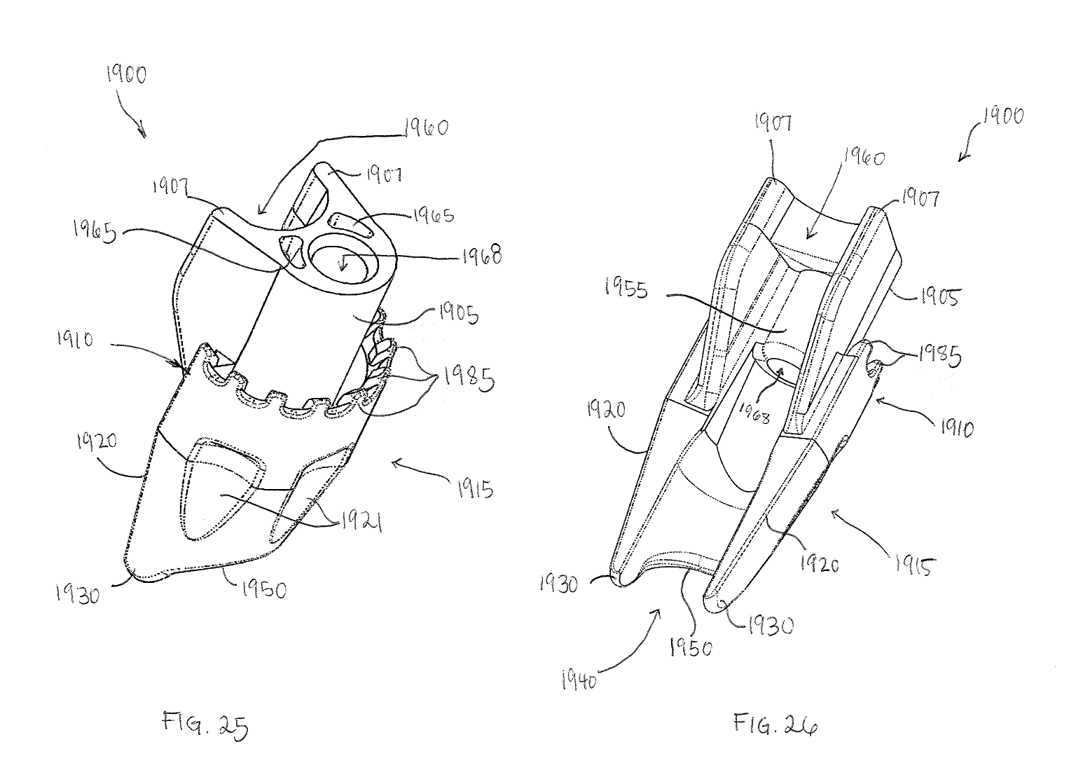

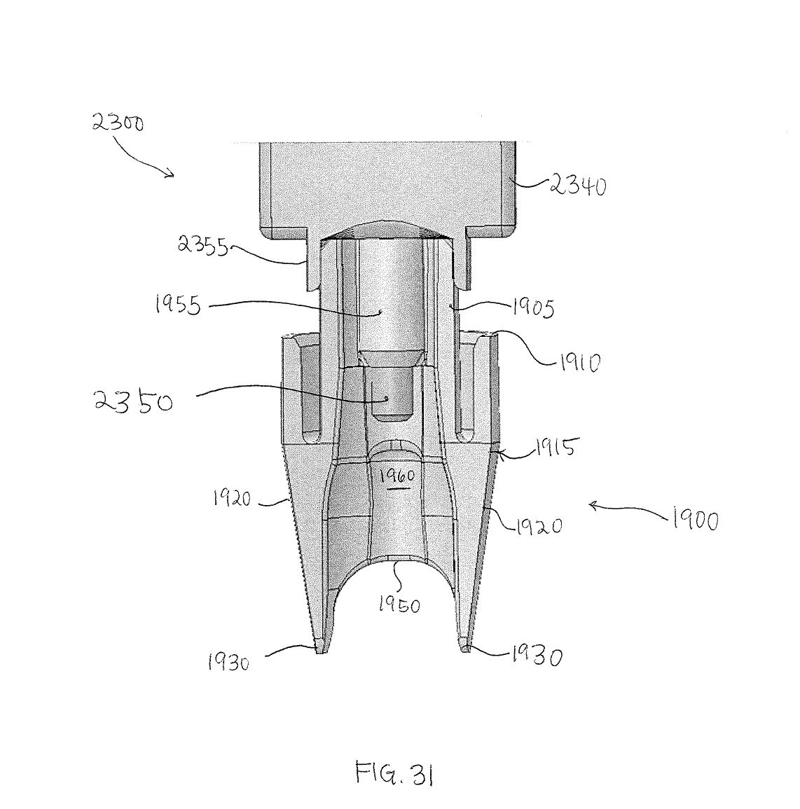

The devices and methods of soft tissue fixation to bone described herein can reduce the burden of surgical repair while optimizing the biological conditions for healing by providing an efficient surgical method. The method can include placing the device 1900 superficial to a segment of soft tissue 2310 superficially juxtaposed to a unicortical blind hole or defect 2308 in a bone 2320 (see FIG. 27). The soft tissue segment 2310 can then be driven with the transiently constricted device 1900 into the blind defect 2308 trapping a portion of the soft tissue segment 2310 between the device 1900 and the bone with a portion of the re-expanded device 1900 disposed subcortically (see FIGS. 23 and 30). The device 1900 can have a longitudinal axis that is oriented in a generally perpendicular alignment relative to the cortical surface and coaxial or parallel with an axis of the bore 2308 through the bone 2320. The device 1900 can be driven using an insertion tool 2300. As best shown in FIG. 23 and also FIG. 31, the insertion tool 2300 can include an elongate element 2340 having a distal end with a tubular receiving element 2355 and a central pin 2350 extending distally therefrom. The device 1900 can be held by the insertion tool 2300 such that a proximal end region of the device 1900 is reversibly coupled to the tubular receiving element 2355 and central pin 2350. The cortical element 1905 can include a tubular feature 1955 having a bore 1968 extending through it from a proximal end to a distal end of the tubular feature 1955 (see FIG. 25). The central pin 2350 can be inserted through the bore 1968 from the proximal end to secure the device 1900 to the insertion tool 2300. The pin can be secured within the bore 1968 via a friction fit. The proximal aspect of the cortical feature 1905 can be received by the tubular receiving element 2355 when the central pin 2350 is inserted through bore 1968. The proximal aspect of the cortical feature 1905 and the tubular receiving element 2355 can additionally or alternatively be coupled together via friction fit. The friction fit of the device 1900 and the insertion tool 2300 can be overcome upon driving the device 1900 into the bone defect 2308 such that the device 1900 remains in place and the insertion tool 2300 can be removed. The insertion tool 2300 can also incorporate a pusher rod type mechanism to release the friction fit between the insertion tool 2300 and the device 1900. It should be appreciated that any of a number of coupling configurations between the insertion tool 2300 and the device 1900 are considered herein. For example, the insertion tool 2300 can include one or more coupling elements configured to engage the proximal end region of the device. In some implementations, the coupling elements can insert or engage with one or more corresponding features 1965 on a proximal end region of the device 1900.

The anchor devices described herein can be constructed of one or more biocompatible materials. In some implementations, one or more portions of the anchor devices, such as the struts, are formed of a biocompatible memory-shaped alloy (e.g. Nitinol, titanium/nickel alloy, nitinol wire mesh) with or without radiolucent material (e.g. PEEK®, Victrex Corp., PolyEtherEtherKetone, or other polymer material). One or more portions of the anchor devices described herein can be formed of ultrahigh molecular weight polypropylene (UHMWPE), Nitinol alloy and others. The anchor devices described herein can be fabricated from absorbable biocompatible polymer(s) such as polyglycolic acid (PGA), and/or polylactic acid (PLA), polydioxanone, and caprolactone. Use of both radiodense and radiolucent elements within the devices provide enhanced mechanical performance while affording improved radiologic monitoring. The anchor devices described herein can incorporate a region composed of bias ply or meshed material (e.g. polymer strand, or wire strand). The struts can be manufactured by laser cutting a nitinol tube as is known in the art. The tubular device can also be manufactured of a material including platinum, gold, palladium, rhenium, tantalum, tungsten, molybdenum, rhenium, nickel, cobalt, stainless steel, Nitinol, and alloys thereof.

The soft tissue structure anchored by the anchor devices described herein can vary. In some implementations, the soft tissue structure is a tendon or a ligament. The anchor devices described herein are particularly suited for use in percutaneous procedures or for use in arthroscopic procedures, including but not limited to biceps tendon, posterior tibial tendon, other relatively narrow and cylindrical tendon repairs, rotator cuff surgery, tendon and ligament affixation or repair, prosthetic attachment, and the like. The anchor devices described herein can be used in any procedure in which it is desired to fix a suture or a soft tissue to a solid object.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

1. A method of fixing soft tissue to bone, comprising:
placing an anchor device superficial to a segment of soft tissue superficially juxtaposed to a unicortical blind hole in a bone, the anchor device having a relaxed state characterized by a first outer dimension;
driving the soft tissue segment into the blind hole using the anchor device, wherein the anchor device transiently constricts to a constricted state during insertion through the blind hole, the constricted state characterized by a second outer dimension that is smaller than the first outer dimension; and
trapping a portion of the soft tissue segment between a portion of the anchor device and a portion of the bone when at least a portion of the anchor device is disposed sub-cortically and relaxes towards the relaxed state.

2.-6. (canceled)

7. An anchor device for attaching a soft tissue segment within bone, the anchor device comprising:
a body having a distal end region and a proximal end region each at least partially surrounding an interior volume forming a slot extending generally along a longitudinal axis of the body; and
a concave saddle surface at the distal end region of the body configured to receive and redirect a soft tissue segment extending through the slot in a first direction from the proximal end region of the body to a second direction that is at an angle to the first direction.

8. The anchor device of claim 7, wherein the distal end region of the body is configured to be positioned sub-cortically to a bone defect and at least a portion of the proximal end region of the body is configured to be positioned intra-cortically within the bone defect.

9. The anchor device of claim 7, wherein during insertion of the anchor device into the bone defect the body is configured to transiently constrict from a relaxed state having a first outer dimension to a constricted state having a second outer dimension that is smaller than the first outer dimension.

10. The anchor device of claim 7, wherein the soft tissue segment is a tendon or a ligament.

11. The anchor device of claim 7, wherein at least a portion of the soft tissue segment is trapped between a portion of the anchor device and a portion of the bone defect when at least a portion of the anchor device is disposed sub-cortically and relaxes towards the relaxed state.

12. The method of claim 7, wherein the anchor device is fabricated from one or more biocompatible polymers.

13.-15. (canceled)

16. An anchor device for attaching a soft tissue segment within bone having a cortex and a sub-cortical region, the anchor device comprising:
a body having a distal end region and a proximal end region each at least partially surrounding an interior volume forming a slot extending generally along a longitudinal axis of the body,
wherein the body is configured to be transiently constricted by a cortical aperture within the bone from a relaxed state having a first outer dimension to a constricted state having a second outer dimension that is smaller than the first outer dimension such that the soft tissue segment is trapped between at least a portion of the body and at least a portion of the bone cortex upon subcortical relaxation of the body towards the relaxed state.

17.-22. (canceled)

23. The anchor device of claim 16, wherein at least a portion of the anchor device flexes in an arcuate manner when in the constricted state.

24. (canceled)

25. The anchor device of claim 16, wherein the body is monolithic and has a substantially conical or frusto-conical shape.

26.-30. (canceled)

31. An anchor device for attaching materials within bone, the anchor device comprising:
a body having a distal end region, a proximal end region, and a plurality of struts extending between the distal end region to the proximal end region and at least partially surrounding an interior volume of the body; and
an attachment feature positioned within the interior volume of the body and coupled near the distal end region, the attachment feature configured to secure material to the body; and
wherein upon removal of a constraint and after delivery of the anchor device into bone the body passively transitions from a constrained, delivery configuration that is radially contracted and axially elongated to a relaxed, deployment configuration that is radially expanded and axially shortened.

32. The anchor device of claim 31, wherein the material secured by the attachment feature to the anchor device is suture or cable material.

33.-39. (canceled)

40. The anchor device of claim 31, wherein the attachment feature comprises a cleat element to secure the material, and wherein a first portion of the material overlaps a second portion of the material resulting in a unidirectional tensioning mechanism of the material with the cleat element.

41. The anchor device of claim 31, wherein the attachment feature comprises a cleat element to secure the material, and wherein the cleat element comprises at least two suture anchor elements, each having an aperture configured to allow the material to extend through.

42. (canceled)

43. The anchor device of claim 41, wherein applying tension to the material forces the at least two suture anchor elements to form a splayed configuration, and wherein the tension applied to the material is maintained by the at least two suture anchor elements wherein at least a part of the material passed through the apertures of the at least two suture anchor elements and wrapped around the commonly formed post results in a portion of the material overlapping another portion of the material.

44. (canceled)

45. The anchor device of claim 31, wherein the material is secured with an interference pin delivered through an opening in the proximal end region of the body.

46. (canceled)

47. The anchor device of claim 31, further comprising a penetrating tip coupled to the distal end region of the body.

48.-51. (canceled)

52. The anchor device of claim 31, wherein the plurality of struts expand near the proximal end region to a greater degree than the plurality of struts expand near the distal end region.

53.-141. (canceled)
